(19) United States
(12) Patent Application Publication
(10) Pub. No.: US 2022/0244271 A1
(43) Pub. Date: Aug. 4, 2022

(54) NON-HLA MARKERS OF TRANSPLANT REJECTION

(71) Applicant: THE REGENTS OF THE UNIVERSITY OF CALIFORNIA, OAKLAND, CA (US)

(72) Inventors: Elaine F. REED, LOS ANGELES, CA (US); Michelle HICKEY, LOS ANGELES, CA (US); Carrie L. BUTLER, LOS ANGELES, CA (US); David W. GJERTSON, LOS ANGELES, CA (US)

(73) Assignee: THE REGENTS OF THE UNIVERSITY OF CALIFORNIA, OAKLAND, CA (US)

(21) Appl. No.: 17/595,015

(22) PCT Filed: May 6, 2020
(86) PCT No.: PCT/US20/31627

§ 371 Date: Nov. 5, 2021

Related U.S. Application Data
(60) Provisional application No. 62/844,027, filed on May 6, 2019.

(51) Int. Cl. G01N 33/68

(52) U.S. Cl. CPC G01N 33/6893; G01N 2800/245

(57) ABSTRACT

The present invention generally relates to transplantation rejection. In particular, the present invention provides compositions, kits, assays, and methods of determining if a subject has allograft rejection, or an increased risk of developing rejection after transplantation, and methods of treatment thereof.

This application claims benefit of U.S. provisional patent application No. 62/844,027, filed May 6, 2019, the entire contents of which are incorporated by reference into this application.

### ACKNOWLEDGEMENT OF GOVERNMENT SUPPORT

This invention was made with government support under Grant Numbers A1042819, A1135201, and DK104687, awarded by the National Institutes of Health. The government has certain rights in the invention.

### FIELD OF THE INVENTION

The present invention generally relates to organ transplantation rejection. In particular, the present invention provides compositions, kits, assays, and methods of determining if a subject has allograft rejection, or an increased risk of developing rejection after transplantation, and methods of treatment. Further, the present invention provides biomarkers, such as non-HLA antibodies, associated with transplantation rejection.

### BACKGROUND

Organ transplantation from a donor to a host recipient is a feature of certain medical procedures and treatment regimes. Following transplantation, immunosuppressive therapy is typically provided to the organ recipient to maintain viability of the donor organ and to avoid graft rejection. When organ transplant rejection occurs, the response is typically classified as a hyperacute rejection, an acute rejection, or a chronic rejection. Hyperacute rejection occurs within minutes to hours following organ transplantation, typically due to antibodies in the recipient's blood stream that react with the new organ, and is usually characterized by widespread glomerular capillary thrombosis and necrosis. Acute rejection (AR) generally occurs in the first 6 to 12 months following organ transplantation, and is a complex immune response that involves T-cell recognition of alloantigen in the graft and an inflammatory response within the graft itself. Chronic rejection is less well-defined than either hyperacute or acute rejection, and is likely due to both antibodies and lymphocytes.

Despite advances in immunosuppressive therapies and transplantation procedures, graft rejection is still a common risk in organ transplant recipients. For example, despite improvements in immunosuppressive therapy over the years, approximately 30-40% of heart transplant recipients require treatment for AR in the first year after transplantation (see Taylor et al., J Heart Transplant., 2009, 28(10):1007-22). Furthermore, AR remains a risk factor for graft dysfunction, mortality, and the development of cardiac allograft vasculopathy (CAV), which is the main cause of late graft failure (see Raichlin et J Heart Lung Transplant,2009, 28(4):320-7).

With advances in human leukocyte antigen (HLA) antibody detection and improved immunosuppression, short-term survival of allografts has increased over the past decade. However, long-term outcomes remain largely unchanged and graft loss due to chronic rejection is a significant problem.

HLA antibodies, particularly donor specific antibodies (DSA), contribute to antibody-mediated rejection (AMR) and acute cellular rejection (ACR) after transplantation. However, a significant proportion of heart transplant patients have been found to have AMR in the absence of HLA or DSA, suggesting that antibodies directed against non-HLA antigens are associated with an increased risk of AMR. Antibodies to non-HLA antigens such as vimenten, MHC class I polypeptide-related sequence A (MICA), and angiotensin II receptor type 1 (AT1R), and antibodies that target the endothelial cell are associated with AMR and chronic allograft vasculopathy after heart transplantation. Additionally, antibodies to non-HLA antigens have been identified and associated with poor outcomes after transplant with other organs.

Endothelial cells (EC) are the first point of contact between the allograft and the transplant recipient's immune system and therefore a potential source of non-HLA antigens that can stimulate a humoral immune response. The endothelial cell crossmatch (ECXM) using primary human aortic EC (HAEC) and the XM-ONE® assay (Olerup SSP AB, Stockholm, Sweden) that employs EC precursors have both been proven clinically relevant to identify patient sera containing antibodies against EC. However, the utility of cell-based assays is limited as they do not identify the antigen that binds the non-HLA antibody present in patient sera. Consequently, the understanding of the breadth of non-H LA antigens that elicit a humoral response leading to poor allograft outcomes is limited by the inability to detect and characterize non-HLA antibodies.

Early detection of AR is one of the major clinical concerns in the care of transplant recipients, including recipients of solid organs such as heart, liver, lung, kidney, and intestines. Detection of AR before the onset of organ dysfunction allows successful treatment of AR with aggressive immunosuppression. It is equally important to reduce immunosuppression in patients who do not have AR to minimize drug toxicity. However, for most organs, rejection can only be unequivocally established by performing a biopsy of that organ.

For example, the current definitive diagnosis of cardiac allograft rejection relies on the endomyocardial biopsy (EMB), an expensive, invasive, and inconvenient procedure. Most heart transplant recipients undergo routine EMB procedures up to 15 times in the first year, and more frequently if rejection is detected. This procedure, however, is limited by sampling error and interobserver variability (see Deng et al., Am J Transplant., 2006, 6(1):150-60; Wong et al., Cardiovasc Pathol., 2005, 14(4):176-80). Potential complications include arterial puncture, vasovagal reactions and prolonged bleeding during catheter insertion, arrhythmias and conduction abnormalities, pneumothorax, biopsy-induced tricuspid regurgitation, and even cardiac perforation (see Baraldi-Junkins et al., J Heart Lung Transplant, 1993, 12(1 Pt 1):63-7; Deckers et al., J Am Coll Cardiol., 1992, 19(1):43-7; Navia et al., J Heart Valve Dis., 2005, 14(2):264-7).

Although the diagnosis of acute rejection can be difficult, detecting immune-related injury in a timely fashion is crucial to ensuring graft health and long-term survival. A noninvasive biomarker panel for acute rejection that allows frequent immunologic monitoring of the graft would be of considerable value (see Evans et al., Am J Transplant., 2005, 5(6):1553-8; Mehra et al., Nat Olin Pract Cardiovasc Med., 2006, 3(3):136-43). Recently, a highly sensitive and specific gene-based biomarker panel was developed for diagnosis and prediction of biopsy confirmed acute renal transplant rejection (see Li et al., Am J Transplant., 2012, 12(10):2710-8; Bromberg et al., Am J Transplant, 2012, 12(10):2573-4), which was independently validated in a randomized multicenter trial (see Chaudhuri et al., Pediatric Transplantation., 2012, 16(5):E183-7; Naesens et al., Am J Transplant.,2012, 12(10):2730-43). The diagnosis of acute rejection prior to development of histopathological changes can enable the optimization of immunosuppressive therapy to prevent progression to chronic allograft dysfunction (see Kienzl et al., Transplantation, 2009, 88(4):553-60).

A noninvasive assay that permits detection of acute graft rejection across different organs with high specificity (to reduce invasive protocol biopsies in patients with low risk of AR) and with high sensitivity (to increase clinical surveillance for patients at high risk of AR), earlier than is currently possible, would result in timely clinical intervention in order to mitigate AR, as well as to reduce the immunosuppression protocols for quiescent and stable patients. Many assays are likely to be dependent upon recipient age, co-morbidities, immunosuppression usage, and/or cause of end-stage renal disease. Therefore, there remains a need for systems and methods for predicting, diagnosing, and monitoring an AR response in a subject that has received an organ transplant.

Further, rejection phenomena are not limited to heart allografts. All organ transplants, including kidney transplants, are subject to rejection (e.g., host versus graft disease). In addition, rejection-like events accompany graft versus host disease (e.g., where transplanted leukocytes and lymphocytes attack the host tissues) and autoimmune disease (e.g., rheumatic fever, in which the heart is the target of an autoantibodies and auto-reactive lymphocytes). In all cases, while aggressive immunosuppression is indicated to reverse or correct the immune reaction, the associated danger of facilitation of opportunistic infections constrains the use of immunosuppression.

Accordingly, there is a need in the art for highly accurate prognostic indicators, and non-invasive assays thereof, of the likelihood of onset of allograft rejection. There is a further need in the art for identifying markers pathologically contributing to or exclusively prognostic indicators of rejection.

The present invention is the first to develop and validate a large high-throughput multiplex bead array to test for the presence of novel and known non-HLA antibodies associated with rejection.

All patents, patent applications, publications, documents, and articles cited herein are incorporated herein by reference in their entireties, unless otherwise stated.

### BRIEF SUMMARY

The present invention provides compositions, assays, kits, or methods of determining if a subject has allograft rejection or subclinical allograft rejection, or an increased risk of suffering from rejection after transplantation.

In some embodiments, the present invention provides a composition comprising a collection of solid-phase substrates coated with one or more homogenous populations of binding agents, wherein each homogenous population of binding agents specifically binds to an antibody that is directed against a single antigen selected from the group consisting of: dexamethasone-induced transcript (DEM, C—X—C motif chemokine ligand 11 (CXCL11), cytokeratin 18 (KRT18), cytokeratin 8 (KRT8), Tubulin, including tubulin alpha 1 b (also referred to as TUBα1b or TUBA1B), latrophilin 1 (LPHN1), Colony stimulating factor 2 (CSF2), Signal Transducer And Activator Of Transcription 6 (STATE), lectin galactoside-binding soluble 3 (LGALS3), SHC Adaptor Protein 3 (SHC3), Glyceraldehyde-3-phosphate dehydrogenase (GAPDH), Glutathione S-Transferase theta-1 (GSTT1), phospholipase A2 receptor 1 (PLA2R1), Interleukin 8 (IL-8), lectin galactoside-binding soluble 8 (LGALS8), Small Nuclear Ribonucleoprotein Polypeptide N (SNPRN), Myosin, Peroxisomal trans-2-enoyl-CoA Reductase (PECR), vimentin (VIM), ATP synthase H+transporting mitochondrial F1 complex beta polypeptide (ATP5B), Collagen H, Prelamin-A/C (LMNA), small nuclear ribonucleoprotein polypeptide B (SNRPB2); fibronectin 1 (FN1), Vinculin (VCL), Thyoglobin (TG), and nephrosis 1 (NPHS1). In some embodiments, the collection of solid-phase substrates further comprises one or more additional homogenous populations of binding agents, wherein each additional homogenous population of binding agents specifically binds to an antibody that is directed against a single additional antigen selected from the group consisting of: Alpha-enolase (ENO1), Agrin (AGRN), Endomucin (EMCN), Sjogren syndrome antigen B (SSB), Actin, fms-related tyrosine kinase 3 ligand (FLT3LG), Protein kinase C eta (PRKCH), and Interleukin 21 (IL-21).

In another embodiment, the compositions of the present invention comprise a collection of one or more distinct homogenous populations of binding agents with each distinct homogenous population of binding agents only being able to specifically bind an antibody that is directed against Tubulin, LPHN1, SNRPN, KRT18, KRT8, LGALS3, SNRPB2, DEXI, Collagen II, GAPDH, PLA2R1, GSTT1, VCL, CSF2, LGALS8, STAT6, TG, IL-8, and SHC3.

In another embodiment, the compositions of the present invention comprise a collection of distinct homogenous populations of binding agents with each distinct homogenous population of binding agents only being able to specifically bind an antibody that is directed against Tubulin, LPHN1, SNRPN, KRT18, KRT8, LGALS3, SNRPB2, DEXI, Collagen II, PLA2R1, GSTT1, VCL, CSF2, LGALS8, and STAT6.

In another embodiment, the compositions of the present invention comprise a collection of distinct homogenous populations of binding agents with each distinct homogenous population of binding agents only being able to specifically bind an antibody that is directed against Tubulin, LPHN1, SNRPN, KRT18, KRT8, LGALS3, SNRPB2, DEXI, Collagen II, PLA2R1, GSTT1, VCL, CSF2, LGALS8, STAT6, IL-8, and SHC3

In another embodiment, the compositions of the present invention comprise a collection of distinct homogenous populations of binding agents with each distinct homogenous population of binding agents only being able to specifically bind an antibody that is directed against Tubulin, LPHN1, SNRPN, KRT18, KRT8, LGALS3, SNRPB2, DEXI, Collagen II, PLA2R1 and GSTT1.

In another embodiment, the compositions of the present invention comprise a collection of distinct homogenous populations of binding agents with each distinct homogenous population of binding agents only being able to specifically bind an antibody that is directed against Tubulin, LPHN1, TG, GAPDH, FN1, NPHS1, VIM, Myosin, VCL and PECR.

In some embodiments, compositions of the present invention comprise a collection of distinct homogenous populations of binding agents with each distinct homogenous population of binding agents only being able to specifically bind an antibody that is directed against DEXI, LGALS3, SNPRN, CSF2, IL-8, STATE, LGALS8, KRT18, KRT8, GSTT1, LMNA, Collagen II, ATP5B, SNRPB2 and PLA2R1.

In some embodiments, compositions of the present invention comprise a collection of distinct homogenous populations of binding agents with each distinct homogenous population of binding agents only being able to specifically bind an antibody that is directed against Tubulin, SHC3 and CXCL11.

In certain embodiments, the solid-phase substrates is porous or non-porous. In certain embodiments, the solid-phase substrates comprise particles, nanoparticles, beads, nanobeads or microspheres. In certain embodiments, the beads are polystyrene beads. In certain embodiments, the collection of solid-phase substrates comprises those that are plate or membrane bound or a microarray. In certain embodiments, the solid-phase substrates are fluorescently labeled, magnetically labeled, chemiluminescent, or radio labeled. In certain embodiments, the solid-phase substrates are labeled with a small molecule. In certain embodiments, the one or more homogenous populations of binding agents are conjugated to the surface of the solid-phase substrates. In certain embodiments, the conjugation is covalent. In certain embodiments, the one or more homogenous populations of binding agents are attached to the surface of the solid-phase substrates by affinity. In certain embodiments, the binding agent is a protein, peptide or polypeptide. In certain embodiments, the solid-phase substrates are coated with one or more different homogenous populations of binding agents that bind to one or more different antigens, and each of the solid-phase substrates is detectably distinguishable from the other solid-phase substrates.

In some embodiments, the present invention provides a method for determining the presence of one or more antibodies in a biological sample obtained from a subject. In some embodiments, the method comprises contacting the biological sample with the composition disclosed herewith, and detecting the binding of the one or more homogenous populations of binding agents to the one or more antibodies. In certain embodiments, the subject is a mammal. In certain embodiments, the subject is a human. In certain embodiments, the subject has received or will receive an organ transplant such as a heart or kidney transplant. In certain embodiments, the heart or kidney transplant is an allograft heart or kidney transplant, respectively. In certain embodiments, the biological sample is blood, plasma, serum, urine, spinal fluid, lymph fluid, synovial fluid, cerebrospinal fluid, tears, saliva, milk, mucosal secretion, effusion, sweat, biopsy aspirates, ascites or fluidic extracts. In certain embodiments, the detecting is by measuring a fluorescence intensity or by an immunological analysis.

In some embodiments, the present invention provides a method for diagnosing a transplant rejection response in a subject that has undergone a heart or kidney transplant. In some embodiments, the method comprises contacting a biological sample obtained from the subject with the composition disclosed herein, and measuring the levels of the one of more antibodies in the sample. In certain embodiments, increased levels of the one or more antibodies, compared to reference levels, indicates that the subject has developed a transplant rejection response in response to the heart or kidney transplant.

In some embodiments, the present invention provides a method for predicting the likelihood of a transplant rejection response in a subject in need of a heart or kidney transplant. In some embodiments, the method comprises contacting a biological sample from the subject with the composition disclosed herein, and measuring the levels of the one or more antibodies in the sample. In certain embodiments, increased levels of the one or more antibodies, compared to reference levels, indicates that the subject has an increased likelihood of developing a transplant rejection response after a heart or kidney transplant.

In some embodiments, the present invention provides a method of treating a subject in need of treatment for a transplant rejection response after receiving a heart or kidney transplant. In some embodiments, the method comprises contacting a biological sample obtained from the subject with the composition disclosed herein, measuring levels of the one or more antibodies in the sample, and administering a treatment for transplant rejection to the subject when there are increased levels of the one or more antibodies, compared to reference levels of the one or more antibodies.

In some embodiments, the present invention provides a kit. In certain embodiments, the kit comprises the compositions disclosed herein, and reagents for detecting the binding of the one or more homogenous populations of binding agents to the antibodies. In other embodiments, the kit further comprises one or more reference samples.

### BRIEF DESCRIPTION OF THE DRAWINGS

FIG. 1 depicts the non-HLA antibodies significantly associated with (A) pediatric and (B) adult renal allograft rejection. A. High throughput multiplex bead analysis was used to identify non-HLA antibodies in sera from pediatric renal transplant patients (n=34 rejection sera, n=95 non-rejection sera). Antibodies to 15 non-H LA antigens (y-axis) were identified to be significantly associated with the time to first rejection with an odds ratio>1 (x-axis). Seven of these, DEXI, CSF2, IL-8, LGALS3, SNPRN, STAT6 and LGALS8, are newly described in relationship to renal transplant rejection. Bars represent the 95% confidence interval (CI). (B) Antibodies to 3 non-HLA antigens (Tubulin, CXCL11, SHC3) were significantly associated with renal allograft rejection in adult renal transplant recipients (n=70 rejection sera, n=90 non-rejection sera). The risk ratios for antibody binding to the remaining non-HLA antigens on the multiplex panel that were not significantly different from one are not shown, * denotes p<0.05, all other non-HLA Ab shown are p<0.1.

FIG. 2 depicts the results of a correlation matrix analysis showing hierarchical clustering of non-HLA antibodies in independent studies of (A) adult cardiac, (B) pediatric renal, and (C) adult renal allograft rejection sera. (A) Non-HLA antibodies associated with cardiac allograft rejection selectively cluster into 4 groups. Two non-HLA antibodies are newly identified to be associated with cardiac allograft rejection in this study (TG and LPHN1). (B) The matrix describes correlation of non-HLA antibodies found in sera of pediatric renal transplant patients with rejection. Non-HLA antibodies associated with renal allograft rejection selectively cluster into 6 groups. Seven non-HLA antibodies are newly identified to be associated with renal allograft rejection in this study (DEXI, CSF2, IL-8, LGALS3, SNPRN, STAT6 and LGALS8). Four non-HLA antibodies cluster independently (PLA2R1, CSF2, GSTT1, and LGALS8). (C) The targets found to be significant in the pediatric renal cohort were used to develop a correlation matrix in the adult renal cohort. Antigens that are independently correlated with rejection are similar between the pediatric and adult renal transplant patients with rejection.

FIG. 3 depicts the results of a correlation matrix analysis showing hierarchical clustering of non-HLA antibodies in a combined analysis of pediatric renal and adult renal allograft rejection sera. The matrices describe correlation of non-HLA antibodies found in sera of rejection patients in a combined analysis of pediatric renal and adult renal sera. Non-HLA antibodies associated with renal allograft rejection selectively cluster into 9 groups. Antibodies to eight non-HLA antigens are newly identified to be associated with renal allograft rejection (LGALS8, SHC3, STAT6, DEXI, IL-8, LGALS3, SNPRN, and CSF2) and 4 non-HLA antigens cluster independently (PLA2R1, STAT6, GSTT1, and CSF2).

FIG. 4 depicts a classification algorithm identifying Non-HLA antibodies that predict renal allograft rejection. Classification and regression tree (CART) analysis showing a binary decision tree that assesses the classification of rejection based on non-HLA antibody strength (MFI). A. CART analysis of sera isolated from adult cardiac allograft transplant patients (n=67 sera). A 1,000 MFI cut point was used in the analysis. The root node, LPHN1, that includes all 67 sera, 49% of which are rejection samples splits at an MFI<1000 into child nodes. As the algorithm progresses to terminal nodes, 65% of rejection samples are correctly identified (far right, dark boxes). Scale bar indicates association with rejection with lighter terminal node boxes correlating to non-rejection. B. CART analysis of sera isolated from pediatric renal allograft transplant patients (n=129 sera). A 1,000 MFI cut point was used in the analysis. The root node, SNPRN, that includes all 129 sera, 26% of which are rejection samples splits at an MFI<1000 into child nodes. As the algorithm progresses to terminal nodes, 56% of non-rejection samples are correctly identified (far left, light boxes). Scale bar indicates association with rejection with lighter terminal node boxes correlating to non-rejection.

FIG. 5 illustrates non-H LA antibodies sorted into 4 groups. The first group are those non-HLA antibodies that were found in multiple transplant cohorts (Core: Tubulin) and all that are predictive of rejection in the CART analysis (FIG. 4). Group 2 are those non-HLA antibodies that sort independently in a correlation matrix and all newly Identified (highlighted with shading, and inclusive all such targets throughout all groups). Two non-HLA antibodies, PLA2R and GSTT1, are found in groups 1 and 2. Group 3 includes those non-H LA antibodies that are found together in correlation matrix analyses and are independent of Groups 1 and 2. Group 4 includes all non-HLA antibodies that are found to be associated with rejection (Table 4).

FIG. 6 illustrates non-HLA antibodies sorted into 4 groups after expanded analysis with cardiac allografts. The first group are those non-HLA antibodies that were found in multiple transplant cohorts (Core: Tubulin) and all that are predictive of rejection in the CART analysis (FIG. 4). Group 2 are those non-HLA antibodies that sort independently in a correlation matrix and all newly Identified (highlighted with shading, and inclusive all such targets throughout all groups). Group 3 includes those non-HLA antibodies that are found together in correlation matrix analyses and are independent of Groups 1 and 2. Group 4 includes all non-HLA antibodies that are found to be associated with rejection (Table 4).

### DETAILED DESCRIPTION

The present invention concerns the diagnosis, prognosis, and/or treatment of acute, chronic, or delayed rejection of heart or kidney transplant. In some embodiments, the invention relates to methods of determining if a subject has an increased risk of developing rejection after transplantation. In some embodiments, the rejection is an acute rejection.

Further, the present invention provides biomarkers (such as protein markers) associated with transplantation. The biomarkers (such as protein markers) described herein can be used in the prediction, diagnosis, prognosis, or treatment of rejection of heart or kidney transplant.

The present invention further encompasses devices for analyzing one or more protein or antibody markers from a subject to determine the presence or absence, or the level of the one or more markers. The presence or absence, or the level of one or more markers is indicative that the subject may have an increased or decreased risk of developing rejection to the organ transplantation compared to a control subject (e.g., a healthy subject who does not express the one or more markers).

The term “collection of solid-phase substrates” refers to a group of substrates that are solid in nature, or can be formed on a solid surface. The term collection means more than one solid substrate, and the number of substrates is determined by the number of distinct markers, such as an antibody, that are being assays according to the methods of the present invention.

The term “homogenous population” refers to a population of molecules that is identical with respect to their molecular structure. In one embodiment, the homogenous population is a collection of a single binding agent that specifically binds to an antibody in a sample, wherein the binding agent has the same amino acid sequence. In another embodiment, the homogenous population is a collection of a single binding agent that specifically binds to an antibody in a sample, wherein the binding agents all have the identical protein structure.

The term “transplantation” or “transplant” refers to the procedure that donor tissue, such as a heart or kidney, is joined with the graft recipient's body.

The term “allogeneic” or “allograft” refers to transplantation of an organ from the same species of animal. However, “xenogeneic” transplants, that is, transplantation of organs from other species of animal into a human, e.g., with hearts or kidneys harvested from transgenic pigs, are also contemplated by the present invention.

The term “rejection” or “transplant rejection” is used herein to refer to the rejection by the immune system of a tissue transplant recipient when the transplanted tissue is immunologically foreign. In specific embodiments, tissue rejection includes but is not limited to, autoimmune organ rejection, e.g., pericarditis, and graft-versus-host mediated rejection. Most frequently, organ rejection occurs following allograft or xenograft transplantation. In some examples, the rejection is an acute rejection. In some examples, the rejection is a chronic rejection.

As used in the art, “acute rejection” is the form of rejection that occurs within the first six months of transplantation, is often mediated by mononuclear cells infiltrating the graft causing acute damage to graft parenchymal cells

As used in the art, “chronic rejection” is the form of rejection that develops within months to years after transplantation. Chronic rejection is the major cause of long-term graft loss.

The term “marker” or “biomarker” is used interchangeably herein to refer to a protein, such as an antibody, that demonstrates altered levels of expression, compared to normal levels, preceding or during heart or kidney transplant rejection. In some embodiments, such proteins are antibodies directed against non-HLA proteins, “non-HLA antibodies,” associated with immune or inflammatory responses. In other embodiments, a marker is a protein found in the tissue of the rejected organ prior to onset of a rejection episode. For example, a marker as used herein is a non-HLA antibody disclosed herein, e.g., in Table 4. In a specific example, the markers of the invention are proteinaceous molecules, and as such, may be modified by the cells that express them. In some cases, partial sequence data confirms that spots having only slight variation in molecular weight, isoelectric point, or both, represent variously modified forms of the same protein. Such modifications include, but are not limited to, glycosylation differences, phosphorylation, N-terminal acetylation, C-terminal amidation, mRNA splicing variations, and the like.

The term “binding agent” refers to a molecule that specifically recognizes and binds to a target molecule of interest. Non-limiting examples of binding agents include any molecules that can form immunocomplexes with a target molecule. For example, in one specific embodiment, the target molecule may be an antibody or antibody fragment, and the binding agent would be, in this particular embodiment, an antigenic molecule, such as but not limited to a polypeptide, to which the antibody or fragment thereof would bind specifically.

The term “antibody” refers to an immunoglobulin molecule or fragment thereof that recognizes and specifically binds to a target, such as a protein, polypeptide, peptide, carbohydrate, polynucleotide, lipid, or combinations of the foregoing through at least one antigen recognition site within the variable region of the immunoglobulin molecule.

The terms “polypeptide,” “peptide,” and “protein” are used interchangeably herein to refer to polymers of amino acids of any length. As used herein, the term polypeptide may refer to a natural or synthetic molecule comprising two or more amino acids linked by the carboxyl group of one amino acid to the alpha amino group of another. In select embodiments, a polypeptide is the binding agent used in the methods and compositions of the present invention. The polypeptides that are used as binding agents in the methods and compositions of the present invention can be of various animal origin, including but not limited to, human, simian, murine, porcine, bovine, canine, equine, ovine, hircine, and cunicular. In certain embodiments, the polypeptide used as the binding agent is a recombinant peptide. The polypeptide can be linear or branched, it can comprise modified amino acids, and it can be interrupted by non-amino acids. The terms also encompass an amino acid polymer that has been modified naturally or by intervention, for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid, or polypeptides comprising one or more conservative substitutions, as well as other modifications known in the art.

The definition of polypeptide of the present invention encompasses antigens or antibodies. For example, the polypeptide can be an antigenic binding agent that specifically binds to the non-HLA antibodies disclosed herein. In some embodiments, the antigenic binding agents comprise the full-length protein or a protein fragment thereof. In some embodiments, the antigenic binding agent comprises or consists of the antigenic determinant thereof. In some embodiments, the antigenic binding agent is of human origin. In other embodiments, the antigenic binding agent is of non-human origin, such as but not limited to simian, murine, porcine, bovine, canine, equine, ovine, hircine, and cunicular.

The term “labeled” as used herein means that the entities comprise a member of a signal producing system and are thus detectable, either directly or through combined action with one or more additional members of a signal producing system. Examples of directly detectable labels include isotopic and fluorescent moieties, often covalently bonded to the solid-phase substrates, the binding agents, and/or the biological samples. The label may include, but is not limited to a fluorescent label, an immunolabel, a magnetic label, a DNA label, a small molecule label, or a radiolabel.

The term “affinity” as used herein means to bind or attach noncovalently. Noncovalent refers to interactions that do not involve the formation of covalent chemical bonds. Noncovalent attachments involve associations between or among molecules and may involve one or more of a variety of noncovalent forces, such as but not limited to, hydrogen bonds, Van der Waals forces, and electrostatic forces. If a ligand has an affinity for a particular target, that means there is a favorable tendency for the ligand to associate specifically and noncovalently with the target to form a complex or complexes. The affinity of a ligand for its target depends on a number of factors, including but not limited to, the conformation of the ligand, the conformation of the target and local environmental parameters such as temperature and ionic conditions, which can strongly influence binding without significantly altering conformation. Non-limiting examples of affinity attachment include the binding between biotin and streptavidin, histidine and nickel, or antibody and antigen.

As used herein, the term “detect” refers to the qualitative or quantitative measurement of undetectable, low, normal, or high concentrations of one or more biomarkers in the biological sample, such as, for example, antigens, antibodies, or other biological molecules.

The term “likelihood of organ rejection” refers to the probability of a rejection episode, which can be predicted based on the level of expression of a marker or markers disclosed herewith.

The term “increased expression” of a marker or markers in a test sample refers to elevated levels of expression of the marker(s) compared to the level of the corresponding marker(s) in a reference sample, or presence of the corresponding marker(s) in a test sample that are not expressed in a reference sample. In some embodiments, the level of a marker as used herein refers to the circulating level of a marker. The term “circulating level” is intended to refer to the amount or concentration of a marker present in a circulating fluid.

Circulating levels can be expressed in terms of, for example, absolute amounts, concentrations, amount per unit mass of the subject, and can be expressed in terms of relative amounts. The level of a marker may also be a relative amount, such as but not limited to, as compared to an internal standard, or baseline levels, or can be expressed as a range of amount, a minimum and/or maximum amount, a mean amount, a median amount, or the presence or absence of a marker. In one example, the increased expression is measured by the median fluorescence intensity (MFI). In other examples, the increased expression is measured by, for example, immunohistochemistry (IHC), enzyme-linked immunosorbent assay (ELISA), or electrochemiluminesence ELISA. In one example, the increased expression refers to the elevated level or the presence of one maker disclosed herein. In one example, the increased expression refers to the level or the presence of a collection of markers disclosed herein.

A “sample,” “test sample,” or “biological sample” as used interchangeably herein is of biological origin, in specific embodiments, such as from a mammal. In certain examples, the sample is a tissue or body fluid obtained from a subject. In other certain examples, the sample is a human sample or animal samples. Non-limiting sources of a sample include blood, plasma, serum, urine, spinal fluid, lymph fluid, synovial fluid, cerebrospinal fluid, tears, saliva, milk, mucosal secretion, effusion, sweat, biopsy aspirates, ascites or fluidic extracts. In a specific example, the sample is a fluid sample. In some embodiments, samples are derived from a subject (e.g., a human) comprising different sample sources described herein.

The term “subject” refers to any animal, e.g., a mammal, including, but not limited to humans and non-human primates, which is to be the recipient of a particular treatment. Typically, as used herein, the terms individual, patient, subject, and “test subject” are used interchangeably and indicate a mammal, in particular a human or non-human primate. In some embodiments, the subject is an adult. In one embodiment, the adult subject is a post-pubescent human. In another embodiment, the subject is a pubescent human. The term “adult” does not include pre-pubescent children. A subject of interest includes one who is to be tested, or has been tested for assessment (e.g., prediction, diagnosis, identification, etc.)

of allograft rejection. The subject may have been previously assessed or diagnosed using other methods, such as those described in current clinical practice. In some embodiments, a subject of interest belongs to a patient sub-population. For example, any of the methods described herein may have use in assessing acute rejection in a patient sub-population with a cardiac or renal allograft rejection (e.g., an acute rejection) score of Grade 0, Grade 1A, Grade 1B, Grade 2, Grade 3A, Grade 3B, or Grade 4. In some embodiments, the subject has a allograft rejection score of ≥Grade 1B. In some embodiments, the subject has at least one histologically proven rejection episode. In some embodiments, the acute cellular rejection (ACR) and antibody-mediated rejection (AMR) are assessed by endomyocardial biopsy (EMB) according to the International Society for Heart and Lung Transplantation (ISHLT) criteria. In some embodiments, the subject has ACR 1R. In some embodiments, the subject has ACR 2R. In some embodiments, the subject has ACR 3R. In some embodiments, the subject has AMR. In some embodiments, the subject has mixed ACR and AMR. In some embodiments, the subject may or may not have had a biopsy, such as a renal or cardiac biopsy. Use of any of the methods, compositions, or kits described herein can non-invasively assess a rejection response in a subject that possibly has a cardiac or renal allograft rejection.

A “reference sample” is used to correlate and compare the results obtained from a test sample. Reference samples can be any biological samples as used herein. The methods of the present invention involve a comparison between levels of one or more markers, e.g., the non-HLA antibodies disclosed herein, in a test sample and a “reference level.” A reference sample can be obtained from various subgroups of individuals, such as but not limited to healthy individuals, individuals who have never received an organ transplant, individuals who have received an organ transplant but have never developed a severe rejection reaction to the transplant and individuals of any age groups. A reference sample also may be obtained from a subject before the subject receives a transplant, or before the subject develops a rejection to transplant. The level of a marker in the “reference sample” is referred to as the “reference level.” Non-limiting examples of reference samples or reference levels are provided below in Example section.

The term “immunological analysis” refers to characterization of the markers disclosed herewith based on immunospecific binding, i.e., reactivity with a specific binding partner of the marker. The paradigm of a specific binding partner is an antigen, in the event that the marker is an antibody. Accordingly, any techniques applicable to antigen-antibody binding extend to binding of any specific binding partner of a marker. Examples of immunological analysis techniques include, but are not limited to immunoblotting, ELISA, radio-immunoassay (RIA), agglutination, immunofluorescence, immunochemiluminescence, immunochromatography, IHC, biosensor, optical sensor, and immunoprecipitation.

Reference to “about” a value or parameter herein includes (and describes) embodiments that are directed to that value or parameter per se. For example, description referring to “about X” includes description of “X.” The term “about” is used to provide flexibility to a numerical range endpoint by providing that a given value may be “a little above” or “a little below” the endpoint without affecting the desired result, but also includes a range of +1-10% of the indicated value. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As used herein, the singular forms “a,” “an,” and “the” include plural forms unless the context clearly dictates otherwise.

Unless defined otherwise, technical and scientific terms used herein have the same meaning as commonly understood by one of ordinary skill in the art to which this invention belongs.

The practice of the present invention will employ, unless otherwise indicated, conventional techniques of protein biology, protein chemistry, molecular biology (including recombinant techniques), microbiology, cell biology, biochemistry, and immunology, which are within the skill of the art. Such techniques are explained fully in the literature, such as “Molecular Cloning: A Laboratory Manual”, second edition (Sambrook et al., 1989); “Current Protocols in Molecular Biology” (Ausubel et al., eds., 1987, periodic updates); “PCR: The Polymerase Chain Reaction”, (Mullis et al., eds., 1994); and Singleton et al., Dictionary of Microbiology and Molecular Biology, 2nd ed., J. Wiley & Sons (New York, N.Y. 1994).

A biomarker of the present invention encompasses a protein, such as an antibody, expressed by cells in subjects undergoing, or having undergone, a heart or kidney transplant rejection reaction. The marker proteins are typically expressed at very low levels, or not expressed in reference samples. In some embodiments, the level of expression of a marker protein increases in association with impending or onset of organ rejection, e.g., an acute rejection.

The present invention provides homogenous populations of binding agents to biomarkers, e.g., non-HLA antibodies, associated with allograft rejection.

In another embodiment, the invention provides non-HLA antibodies that are predictive of allograft rejection, including but not limited to Tubulin, LPHN1, SNRPN, KRT18, KRT8, LGALS3, SNRPB2, DEXI, Collagen II, PLA2R1, GSTT1, VCL, CSF2, LGALS8, STAT6, GAPDH, IL-8, SHC3, ENO1, AGRN, EMCN, and SSB. In some embodiments, any one, or any combination of two or more, of the forgoing non-HLA antibodies are informative as markers of allograft rejection. In some embodiments, a combination of non-HLA antibodies that are informative of allograft rejection based on additional organ-specific analysis includes Tubulin, LPHN1, SNRPN, KRT18, KRT8, DEXI, and GAPDH. In some embodiments, assessing the levels of any one, or any combination of two or more, of Tubulin, LPHN1, SNRPN, KRT18, KRT8, LGALS3, SNRPB2, DEXI, Collagen II, PLA2R1, GSTT1, VCL, CSF2, LGALS8, STAT6, GAPDH, IL-8, SHC3, ENO1, AGRN, EMCN, and SSB non-HLA antibodies is predictive of allograft rejection. In some embodiments, a combination of non-HLA antibodies that are informative of allograft rejection, for example, based on additional organ-specific analysis, includes Tubulin, LPHN1, SNRPN, KRT18, KRT8, DEXI, and GAPDH.

In some embodiments, any one, two, three, four, five, six, seven, eight, nine, ten, 11, 12, 13, 14 or 15 of the Tubulin, LPHN1, SNRPN, KRT18, KRT8, LGALS3, SNRPB2, DEXI, Collagen II, PLA2R1, GSTT1, VCL, CSF2, LGALS8, and STAT6 non-HLA antibodies are informative as independent markers of allograft rejection. In some embodiments, any one, two, three, four, five, six, or seven additional non-HLA antibodies, of GAPDH, IL-8, SHC3, ENO1, AGRN, EMCN, and SSB, are informative as independent markers of allograft rejection. In some embodiments, assessing the levels of any one, two, three, four, five, six, seven, eight, nine, ten, 11, 12, 13, 14 or 15 of the Tubulin, LPHN1, SNRPN, KRT18, KRT8, LGALS3, SNRPB2, DEXI, Collagen II, PLA2R1, GSTT1, VCL, CSF2, LGALS8, and STAT6 non-HLA antibodies are predictive of allograft rejection. In some embodiments, assessing the levels of any one, two, three, four, five, six, or seven of the GAPDH, IL-8, SHC3, ENO1, AGRN, EMCN, and SSB non-HLA antibodies are predictive of allograft rejection.

In some embodiments, assessing the levels of any one, two, three, four, five, six, seven, eight, nine or ten of the Tubulin, LPHN1, TG, GAPDH, FN1, NPHS1, VIM, Myosin, VCL and PECR non-HLA antibodies are predictive of cardiac allograft rejection. For example, assessing levels of TG and LPHN1 non-HLA antibodies is predictive of cardiac allograft rejection. In another example, assessing levels of Tubulin, LPHN1, TG and VCL non-HLA antibodies is predictive of cardiac allograft rejection. In another example, assessing levels of Tubulin, LPHN1 and TG non-HLA antibodies is predictive of cardiac allograft rejection. In another example, assessing levels of DEXI, EMCN, SNRPN, LPHN1 and SSB non-HLA antibodies is predictive of cardiac allograft rejection. In another example, assessing levels of KRT18, GAPDH, AGRN, ENO1 and EMCN non-HLA antibodies is predictive of non-rejection. In some embodiments, assessing the levels of any one, two, three, four, five, six, seven, eight, nine, ten, eleven, twelve, thirteen, fourteen, fifteen, sixteen, seventeen, or eighteen of the ENO1, AGRN, EMCN, SSB, Actin, FLT3LG, PRKCH, IL-21, Tubulin, LPHN1, TG, GAPDH, FN1, NPHS1, VIM, Myosin, VCL and PECR non-HLA antibodies are predictive of cardiac allograft rejection.

In some embodiments, assessing the levels of any one, two, three, four, five, six, seven, eight, nine, ten, 11, 12, 13, 14 or 15 of the DEXI, LGALS3, SNPRN, CSF2, IL-8, STAT6, LGALS8, KRT18, KRT8, GSTT1, LMNA, Collagen II, ATP5B, SNRPB2 and PLA2R1 non-HLA antibodies are predictive of pediatric renal allograft rejection.

For example, assessing levels of DEXI, LGALS3, SNPRN, CSF2, IL-8, STAT6 and LGALS8 non-HLA antibodies is predictive of renal allograft rejection. In another example, assessing levels of DEXI, LGALS3, SNPRN, CSF2, STAT6, LGALS8, KRT18, KRT8, GSTT1, Collagen II, SNRPB2, and PLA2R1 non-HLA antibodies is predictive of renal allograft rejection. In another example, assessing levels of Tubulin, DEXI, LGALS3, SNPRN, KRT18, KRT8, GSTT1, Collagen II, SNRPB2 and PLA2R1 non-HLA antibodies is predictive of renal allograft rejection.

In some embodiments, assessing the levels of any one, two or three of the Tubulin, SHC3 and CXCL11 non-HLA antibodies are predictive of adult renal allograft rejection.

In certain embodiments, the invention provides a composition for determining the presence of one or more antibodies in a biological sample. In certain embodiments, the composition comprises a collection of solid-phase substrates coated with one or more homogenous populations of binding agents. In certain embodiments, each of the homogenous population of binding agents specifically binds to a non-HLA antibody disclosed herein.

In some embodiments, the coating is by conjugation, i.e., the one or more homogenous populations of binding agents are conjugated to the surface of the solid-phase substrates. In some embodiments, the conjugation is covalent. In some embodiments, the coating is by covalent attachment. Example of covalent attachment includes but is not limited to glutaraldehyde. In some embodiments, the coating is by covalent crosslinking. Examples of covalent attachment include but are not limited to 1-ethyl-3-(3-dimethylaminopropyl)carbodiimide hydrochloride (EDC), H-benzotriazol-1-yloxytris (dimethylamino) phosphonium hexafluorophosphate (BOP), N-ethoxycarbonyl-2-ethoxy-12-dihydroquinoline (EEDQ), (1-[3-(dimethylamino)propyl]-3-ethylcarbodiimide hydrochloride) (EDAC), and N-hydroxysuccinimide (NHS). In some embodiments, the coating is by physical adsorption. In some embodiments, the coating is by encapsulation. Examples of encapsulation include but are not limited to polymers. In some embodiments, the coating is by affinity attachment. Examples of physical adsorption include but are not limited to biotin/streptavidin, histidine/nickel, and antibody/antigen. Methods for covalent attachments, covalent crosslinking, physical adsorption, encapsulation, and affinity attachment are generally known in the art and are encompassed by the present invention.

In some embodiments; the solid-phase substrates comprise particles, nanoparticles; beads, nanobeads, or microspheres. In some embodiments, the solid-phase substrates can be porous or nonporous. In some embodiments, the substrate can be array-based. In another embodiment, a solid-phase substrate of the present invention comprises a magnetic-based protein assay component. In other embodiments, the substrate can be organic or inorganic; can be metal (e.g., copper or silver) or non-metal; can be a polymer or nonpolymer; can be conducting, semiconducting or nonconducting (insulating); can be reflecting or nonreflecting; etc. For example, the substrate can comprise polyethylene, polytetrafluoroethylene, polystyrene, polyethylene terephthalate, polycarbonate, gold, silicon, silicon oxide, silicon oxynitride, indium, tantalum oxide, niobium oxide, titanium, titanium oxide, platinum, iridium, indium tin oxide, diamond or diamond-like film, etc.

Substrates as described above can be formed of any suitable material, including but not limited to a material selected from the group consisting of metals, metal oxides, alloys, semiconductors, polymers (such as organic polymers in any suitable form including woven, nonwoven, molded, extruded, cast, etc.), silicon, silicon oxide, ceramics, glass, and composites thereof.

In certain embodiments, the solid-phase substrates are labeled. In certain embodiments, the binding agents are labeled. In certain embodiments, the biological samples are labeled. In some embodiments, any one or two components of the above are labeled. In some embodiments, all of the above are labeled.

In one embodiment, the label is a fluorescent moiety. Fluorescent moieties or labels of interest include, but are limited to, coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum Dye™, fluorescent energy transfer dyes, such as thiazole orange-ethidium heterodimer, TOTAB, etc. In one embodiment, the fluorescent label is a phycoerythrin (e.g., R-phycoerythrin (R-PE)). R-PE exhibits extremely bright red-orange fluorescence with high quantum yields. It is excited by laser lines from 488 to 561 nm, with absorbance maxima at 496, 546, and 565 nm and a fluorescence emission peak at 578 nm. R-PE is a large molecule used for fluorescence-based detection, such as flow cytometry, microarray assays, ELISAs, and other applications that require high sensitivity but not photostability.

Fluorescent dyes can be detected in droplets in real time with high resolution, and the availability of many fluorescent dyes with distinct excitation and emission wavelengths allow monitoring many labels in one experiment. Sets of fluorescent dyes can be selected so as to allow for a simultaneous detection of more than one dye in the same reaction. A set of dyes that can be detected at the same time can include, but are not limited to, Cy3, Cy5, FAM, JOE, TAMRA, ROX, dR110, dR6G, dTAMRA, dROX, or any mixture thereof. Any of those dyes can be used individually or in any combination to practice an embodiment herein. A dye can allow for single molecule detection. A large number of fluorescent dyes have been synthesized, and are commercially available in different formats.

In some embodiments, the label is an affinity tag. Common choices for affinity tags are known in the art, such as biotin, histidine, Glutathione S-transferase (GST), and maltose-binding protein (MBP). Antibody and antigen can also be used as affinity tags. In one specific embodiment, the binding agent is labeled with an affinity tag, and this label is used to coat the binding agent onto the solid substrate.

In some embodiments, the label is an isotopic moiety. For example, the isotopic moiety comprises ³²P, ³³P, ³⁵S, ¹²⁵I, and the like. In some embodiments, the solid-phase substrates are magnetically labeled. In some embodiments, the solid-phase substrates are labeled with one or more small molecules.

In some embodiments, each solid phase substrate is detectably distinguishable from other solid phase substrates within the composition. In some embodiments, the detectably distinguishable solid phase substrates are distinguishable by labeling.

Described herein, in one embodiment, is a method for detecting biomarkers of solid organ graft rejection in a sample from a patient. In some embodiments, the method comprises: (a) detecting in a sample obtained from the patient a first graft rejection biomarker, wherein the first graft rejection biomarker is an antibody directed against Tubulin, and one or more additional graft rejection biomarkers, wherein the additional graft rejection biomarker is an antibody that is directed against an antigen selected from the group consisting of: LPHN1, SNRPN, KRT18, KRT8, LGALS3, SNRPB2, DEXI, Collagen II, PLA2R1, GSTT1, VCL, CSF2, LGALS8, STAT6, GAPDH, IL-8, SHC3, ENO1, AGRN, EMCN, and SSB; (b) determining whether the amount of graft rejection biomarker is significantly different from the amount in a control sample; and (c) detecting biomarkers of graft rejection when the determining in (b) shows significant difference in the patient sample relative to the control sample. In other embodiments, the first graft rejection biomarker is an antibody directed against Tubulin, and the additional graft rejection biomarker is an antibody directed against an antigen selected from the group consisting of LPHN1, SNRPN, KRT18, KRT8, LGALS3, SNRPB2, DEXI, Collagen II, PLA2R1, GSTT1, VCL, CSF2, LGALS8, STAT6, GAPDH, IL-8, SHC3, ENO1, AGRN, EMCN, and SSB. In some embodiments, the detecting further comprises detecting in the patient sample one or more additional graft rejection biomarkers selected from the group consisting of antibodies directed against TG, PECR, NPHS1, FN1, Myosin, VIM, ATP5B, LMNA, CXCL11, Actin, FLT3LG, PRKCH, and IL-21.

Described herein, in another embodiment, is a method for detecting biomarkers of solid organ graft rejection in a sample from a patient. In some embodiments, the method comprises: (a) detecting in a sample obtained from the patient a first graft rejection biomarker, wherein the first graft rejection biomarker is an antibody directed against dexamethasone-induced transcript (DEXI), and one or more additional graft rejection biomarkers, wherein the additional graft rejection biomarker is an antibody that is directed against an antigen selected from the group consisting of C—X—C motif chemokine ligand 11 (CXCL11), cytokeratin 18 (KRT18), cytokeratin 8 (KRT8), TUBα1b: tubulin alpha 1 b (TUBA1B), and Tubulin; (b) determining whether the amount of graft rejection biomarker is significantly different from the amount in a control sample; and (c) detecting biomarkers of graft rejection when the determining in (b) shows significant difference in the patient sample relative to the control sample. In other embodiments, the first graft rejection biomarker is an antibody directed against Tubulin, and the additional graft rejection biomarker is an antibody directed against an antigen selected from the group consisting of CXCL11, DEXI, KRT18, and KRT8.

In some embodiments, the detecting further comprises detecting in the patient sample one or more additional graft rejection biomarkers selected from the group consisting of antibodies directed against LPHN1, CSF2, STAT6, LGALS8, SHC3, GAPDH, GSTT1, and PLA2R1. In other embodiments, the detecting further comprises detecting in the patient sample one or more additional graft rejection biomarkers selected from the group consisting of antibodies directed against LPHN1, CSF2, STAT6, LGALS8, SHC3, GAPDH, GSTT1, PLA2R1, IL-8, LGALS3, SNPRN, Myosin, PECR, VIM, ATP5B, Collagen II, LMNA, and SNRPB2.

In some embodiments, any one, two, three, four, five, six, seven, eight, nine, ten, 11, 12, 13, 14, 15, 16, 17, 18, or 19, of the non-HLA antigenic targets disclosed herein are informative as independent markers of cardiac allograft rejection. In some embodiments, any one, two, three, four, five, six, seven, eight, nine, ten, 11, 12, 13, 14, 15, 16, 17, 18, or 19 of the non-HLA antigenic targets disclosed herein predictive of cardiac allograft rejection.

In some embodiments, any one, two, three, four, five, six, seven, eight, nine, ten, 11, or 12 of the non-HLA antigenic targets disclosed herein are informative as independent markers of renal allograft rejection. In some embodiments, any one, two, three, four, five, six, seven, eight, nine, ten, 11, or 12 of the non-HLA antigenic targets disclosed herein predictive of renal allograft rejection.

In one embodiment, the method is performed with 8 or fewer graft rejection markers. Optionally, the detecting can be performed with up to 5, 10, 15, 20, 25, 30, or up to 35 graft rejection markers. In some embodiments, the graft rejection markers are selected exclusively from the group consisting of antibodies directed against DEXI, CXCL11, KRT18, KRT8, TUBA1B, Tubulin, LPHN1, CSF2, STAT6, LGALS8, SHC3, GAPDH, GSTT1, PLA2R1, IL-8, LGALS3, SNPRN, Myosin, PECR, VIM, ATP5B, Collagen II, LMNA, SNRPB2, VCL, TG, FN1, ENO1, AGRN, EMCN, SSB, Actin, FLT3LG, PRKCH, and IL-21, as well as combinations of two of more of these markers. In some embodiments, the graft rejection markers are selected exclusively from the group consisting of antibodies directed against DEXI, CXCL11, KRT18, KRT8, TUBA1B, Tubulin, LPHN1, CSF2, STAT6, LGALS8, SHC3, GAPDH, GSTT1, PLA2R1, IL-8, LGALS3, SNPRN, Myosin, PECR, VIM, ATP5B, Collagen II, LMNA, SNRPB2, as well as combinations of two of more of these markers. In other embodiments, the graft rejection markers further include one or more additional markers beyond those listed here, such as further markers of interest to the user.

Additionally provided is a method for assaying a combination of markers in a sample of biological fluid obtained from a human subject, the method comprising performing an immunoassay by contacting the sample with the solid support of a kit or composition as described herein. Examples of immunoassays include, but are not limited to, an enzyme-linked immunosorbent assay (ELISA), and a bead-based, particle-based, or other multiplex assay.

In some embodiments, the sample is plasma or serum. In some embodiments, the method further comprises contacting the sample with the conjugates of the kit, and assaying the reaction of the conjugates with the sample. In some embodiments, the method further comprises contacting the antigen standards with the solid support and the conjugates, and assaying the relative levels of graft rejection biomarkers in the sample relative to the antigen standards.

Further provided is a method of assaying graft rejection biomarkers in a sample of serum or plasma. In some embodiments, the method comprises: (a) providing a binding agent that specifically binds to an antibody that is directed against DEXI and one or more binding agents that specifically bind to an antibody that is directed against a single antigen selected from the group consisting of CXCL11, KRT18, KRT8, TUBA1B, and Tubulin; (b) providing a microtiter plate coated with the binding agents; (c) adding the serum or plasma to the microtiter plate; (d) providing alkaline phosphatase-antibody conjugates reactive with graft rejection biomarkers to the microtiter plate; (e) providing p-nitrophenyl-phosphate to the microtiter plate; and (f) assaying the reaction which occurs as a result of steps (a) to (e) relative to a standard curve to determine the levels of graft rejection biomarkers in the sample.

In some embodiments, the detecting further comprises detecting in the patient sample one or more additional graft rejection biomarkers selected from the group consisting of antibodies directed against LPHN1, CSF2, STAT6, LGALS8, SHC3, GAPDH, GSTT1, PLA2R1, IL-8, LGALS3, SNPRN, Myosin, PECR, VIM, ATP5B, Collagen II, LMNA, SNRPB2, VCL, TG, FN1, ENO1, AGRN, EMCN, SSB, Actin, FLT3LG, PRKCH, and 1L-21. In some embodiments, the detecting further comprises detecting in the patient sample one or more additional graft rejection biomarkers selected from the group consisting of antibodies directed against LPHN1, CSF2, STAT6, LGALS8, SHC3, GAPDH, GSTT1, and PLA2R1. In some embodiments, the detecting further comprises detecting in the patient sample one or more additional graft rejection biomarkers selected from the group consisting of antibodies directed against LPHN1, CSF2, STAT6, LGALS8, SHC3, GAPDH, GSTT1, PLA2R1, IL-8, LGALS3, SNPRN, Myosin, PECR, VIM, ATP5B, Collagen II, LMNA, and SNRPB2.

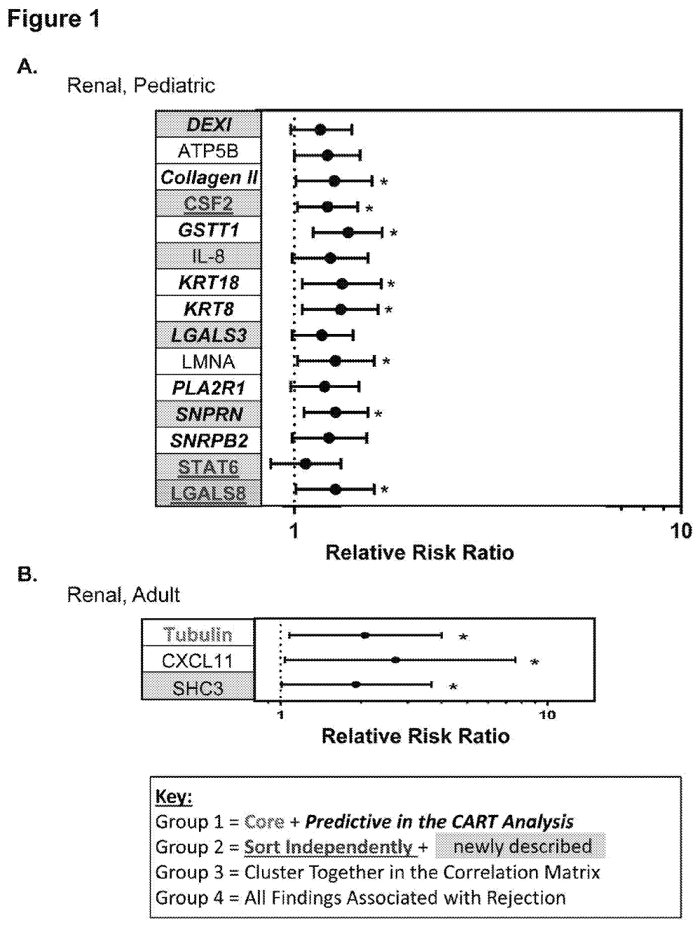

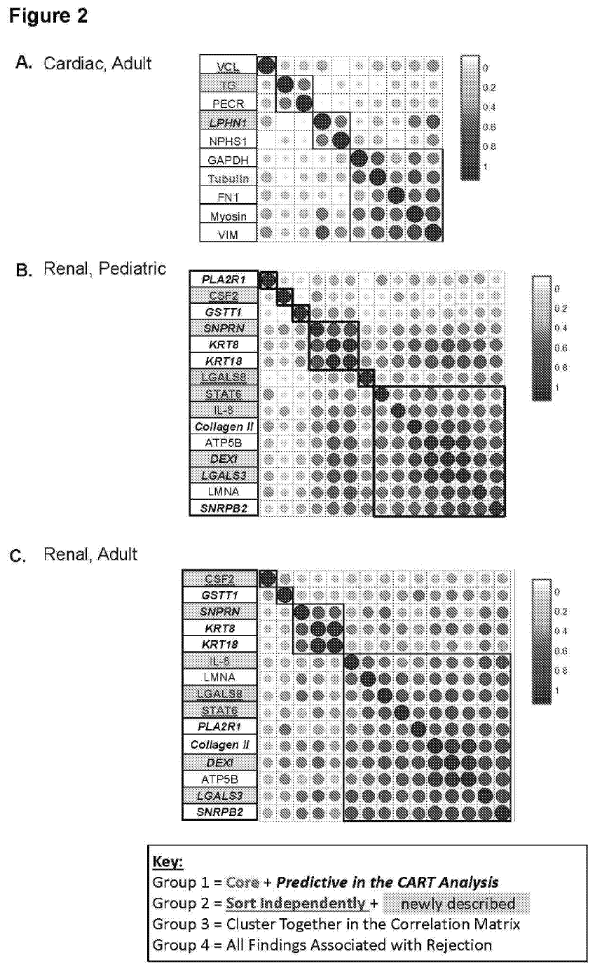

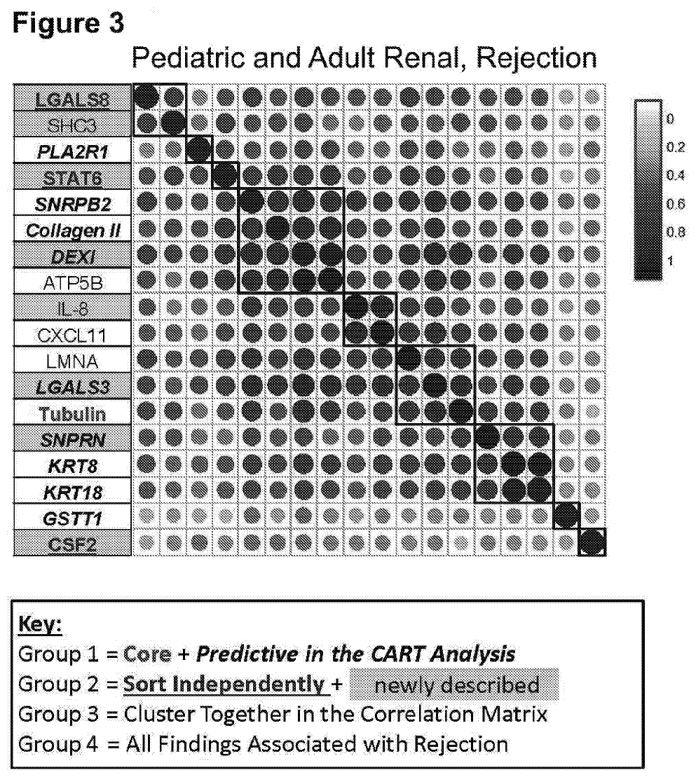

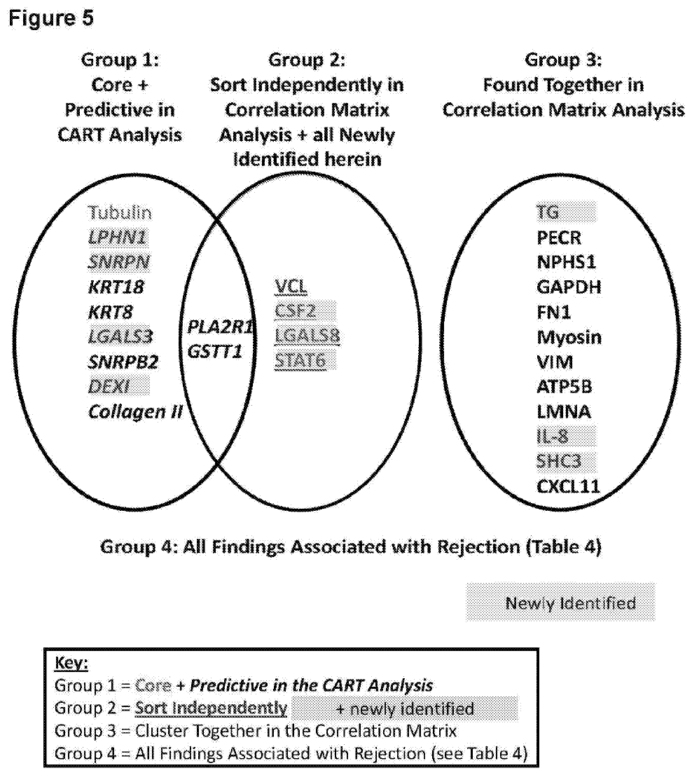

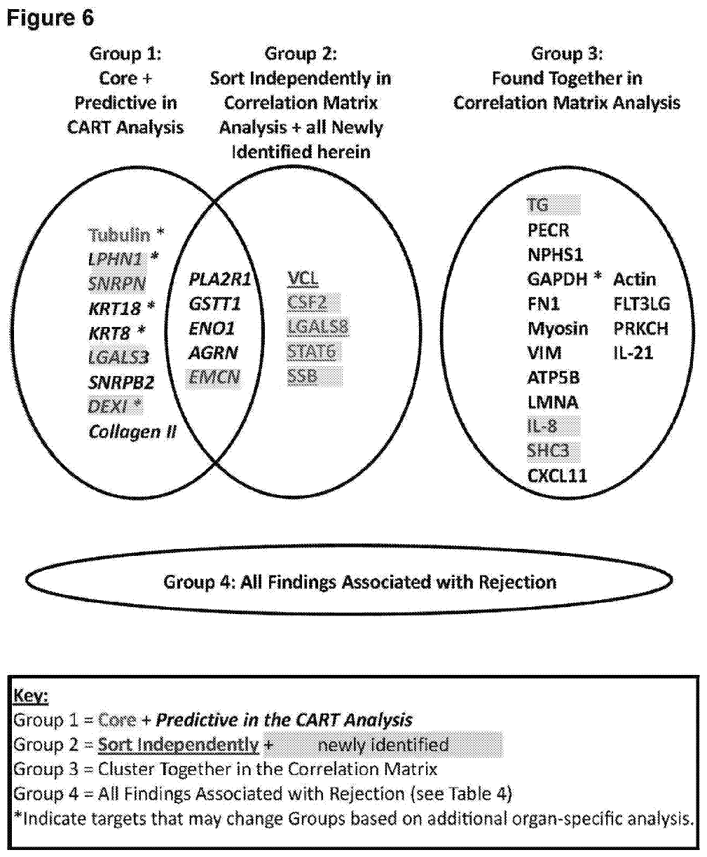

Other groupings of graft rejection biomarkers can be identified by reference to the Figures and Tables herein. For example, a selection of biomarkers for use together comprises one or more members of those markers identified in FIG. 5 or FIG. 6 as “Group I”, “Group II”, and/or “Group III”, and/or identified in Table 4, and grouping one or more members of a group together. In another example, a selection comprises one or more representatives of differing groups of those identified in FIG. 5 or FIG. 6 and Table 4. A representative grouping of biomarkers for use together can comprise one or more members of one of the columns presented in Table 3 and/or Table 5, thereby tailoring the grouping to detection of cardiac graft rejection; or adult renal graft rejection, or pediatric renal graft rejection. In another example, a “core” biomarker (e.g., Tubulin) that is associated with rejection across differing organ systems and populations is selected and combined with one or more biomarkers associated with each of the columns identified in Table 3 and/or Table 5. Other bases for selecting biomarkers for use together include, but are not limited to, whether the biomarkers are predictive in CART analysis (classification and regression tree analysis), whether the biomarkers sort independently in analyses that indicate they are independent predictors of rejection, whether the markers cluster together (or independently, as in VCL) in the correlation matrix (see FIGS. 2 and 3), whether the biomarkers were significantly associated with the time to first rejection (see FIG. 1), whether the biomarkers are newly described herein (Table 1 or Table 6) or had previously been associated with graft rejection, Various other combinations of biomarker groupings are also contemplated. Some representative examples include, but are not limited to, a selected individual biomarker, such as antibody directed against Tubulin or DEXI or EMCN or LGALS3 or SNPRN or LPHN1 or SSB or TG or CXCL11 or KRT8 or KRT18 as suggested individual examples, in combination with one, two, three, four, five, or more additional biomarkers; selected subsets of markers grouped together herein (e.g., as grouped in Groups I, II, III or IV, or in one of the Tables or Figures herein); selected combinations that include one or more representative markers within such subsets.

In some embodiments, the steps recited above are performed for 2, 3, 4, 5, 6, 7, 8, 9, 10, 12, 15, 20, 25, 30, or all 31 of the markers listed in Table 1. In one embodiment, the set of markers consists of 8 or fewer markers listed in Table 1, In another embodiment, the set of markers consists of 6 or fewer markers listed in Table 1. In yet another embodiment, the set of markers consists of 4 or fewer markers listed in Table 1. Representative groupings of biomarkers of graft rejection include, but are not limited to, antibodies directed against DEXI, KRT8, and KRT18; CXC11 and Tubulin/TUBA1B; DEXI, KRT8, KRT18, CXC11, and Tubulin/TUBA1B; DEXI, SNPRN, Smith Antigen, LGALS3, ANXA2R; Jo-1, CCP, and TG; DEXI, LPHN1, CSF2, LGALS8, STATE, and SHC3. Other groupings include Tubulin, LPHN1, SNRPN, KRT18, KRT8, DEXI, and GAPDH, as well as various groupings identified in FIGS. 5 and 6, and in the various exemplary embodiments listed herein.

In one embodiment, the present invention describes a method for determining the presence of one or more of non-HLA antibodies disclosed herein in a biological sample obtained from a subject, for example, a subject who has received an organ transplant or is going to receive an organ transplant. Using such methods provided herein, one could screen or monitor antibodies to non-HLA antigens in cardiac and/or renal allograft patients, and thus assess their risk of developing a rejection.

The first step of methods generally involves contacting a biological sample obtained from a subject with the composition disclosed herein.

Contacting the biological sample with the composition is generally a matter of adding the composition to the sample, or vice versa, and incubating the mixture for a period of time long enough for the composition to specifically bind to any target antibodies present in the sample. Effective or optimal conditions can be determined using methods known in the art.

Any convenient protocol for obtaining such biological samples may be employed, where suitable protocols are well known in the art. When obtaining a sample from a subject (e.g., blood sample), the amount can vary depending upon subject size and the condition being screened. In some aspects, up to about 1, 2, 3, 4, 5, 6, 7, 8, 9, 10, 20, 30, 40, or 50 mL of a sample is obtained. In some aspects, about 1-50, 2-40, 3-30, or 4-20 mL of sample is obtained. In some aspects, more than about 5, 10, 15, 20, 25, 30, 35, 40, 45, 50, 55, 60, 65, 70, 75, 80, 85, 90, 95 or 100 mL of a sample is obtained. In some aspects, up to about 1, 2, 3, 4, 5, 6, 7, 8, 9, 10, 20, 30, 40, or 50 μL of a sample is obtained. In some aspects, about 1-50, 2-40, 3-30, or 4-20 μL of sample is obtained. In some aspects, more than about 5, 10, 15, 20, 25, 30, 35, 40, 45, 50, 55, 60, 65, 70, 75, 80, 85, 90, 95 or 100 μL of a sample is obtained. In some aspects, less than about 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 pg, 5 pg, 10 pg, 20 pg, 30 pg, 40 pg, 50 pg, 100 pg, 200 pg, 500 pg, 1 mg, 5 mg, 10 mg, 50 mg, 100 mg, 200 mg, 500 mg, 1 gram, 5 grams, 10, grams, 20 grams, 50 grams, 100 grams or more of sample are obtained from the sample for analysis. In some aspects, about 1-5 pg, 5-10 pg, 10-100 pg, 100 pg-1 ng, 1-5 ng, 5-10 ng, 10-100 ng, or 100 ng-1 pg of sample are obtained from the sample for analysis. In some aspects, about 1 mg of sample are obtained from the sample for analysis.

A plurality of biological samples may be collected at any one time. A biological sample or samples may be taken from a subject at any time, including before transplantation, at the time of transplantation, or at any time following transplantation.

Following contacting a biological sample with a composition described herein, a signal is generated from the contacting step that can be detected using any suitable method known in the art. Exemplary methods can include, but are not limited to, visual detection, fluorescence detection (e.g., fluorescence microscopy), scintillation counting, surface plasmon resonance, ellipsometry, atomic force microscopy, surface acoustic wave device detection, autoradiography, and chemiluminescence. As one of skill in the art will appreciate, the choice of detection method will depend on the specific labeling agent employed.

In some embodiments, the detecting is carried out by measuring a fluorescence intensity. Such methods are generally known in the art. For example, the xMAP Technology by Luminex (Luminex Corp., Austin, Tex.) allows one to perform up to 500 immunoassays in varying combinations in a single reaction in a standard 96-well microplate. In some embodiments, the detecting is carried out by an immunological analysis. The Example section provides exemplary embodiments of the effective or optimal conditions for the methods described herein.

In some embodiments, the detecting is carried out by labeled secondary anti-human antibody. Labeled secondary anti-human antibodies are commonly used in the art and available from various vendors. In some embodiments, the secondary anti-human antibodies are labeled by enzyme conjugates. Non-limiting examples include alkaline phosphatase (AP) or horseradish peroxidase (HRP). In some embodiments, the secondary anti-human antibodies are labeled by fluorescent conjugates. Non-limiting examples include fluorescein (FITC), tetramethylrhodamine (TRITC), Alexa Fluor, phycoerythrin, etc. In some embodiments, the secondary anti-human antibodies are labeled by biotin. Specific embodiments of labeled secondary anti-human antibody are provided in the Example section.

In certain embodiments, the present invention provides methods for diagnosing transplant rejection responses in a subject that has undergone a heart or kidney transplant. In yet another embodiment, the present invention describes a method for predicting the likelihood of a transplant rejection response in a subject in need of a heart or kidney transplant.

In specific embodiments, the methods provided herein comprise measuring the binding of the one or more homogenous populations of binding agents to the one or more antibodies that may be present in the sample. In some embodiments, the methods comprise measuring the levels of the one of more antibodies in the sample. In some embodiments, increased levels of the one or more antibodies, compared to reference levels, indicates that the subject has a likelihood of developing a transplant rejection response after a heart or kidney transplant.

In some embodiments, whether the subject will have a rejection (e.g., an acute rejection) response is determined based upon the presence of one or more of the biomarkers disclosed herein. In some embodiments, the presence of one or more of the biomarkers comprise any one, two, three, four, five, six, seven, eight, nine, ten, 11, 12, 13, 14, 15, 16, 17, or 18 of the non-HLA antibodies disclosed herein at levels above reference levels are informative as independent markers of allograft rejection.

The accuracy of the methods of diagnosis and/or prognosis can be measured by the degree of closeness of a measured or calculated value to its actual value. For instance, the accuracy of the methods provided herein can be measured by the proportion of correctly predicted rejection or non-rejection. In some embodiments of the present invention, the accuracy of the methods described herein is the number of subjects without rejection that are predicted by the methods described herein to not have rejection divided by the total number of subjects who actually do not have rejection. In other embodiments, the accuracy of the methods described herein is the number of subjects predicted by the methods described herein to have rejection divided by the total number of subjects who actually have rejection. In some embodiments, the methods described herein comprises assessing (e.g., predicting, diagnosing, identifying, etc.) a rejection response with an accuracy of about 60-100%. In some embodiments, the accuracy is about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, but no more than 100%. In some embodiments, the accuracy is about 60-65%, 65-70%, 70-75%, 75-80%, 80-85%, 85-90%, 90-95%, or 95-100%, but no more than 100%. In some embodiments, the accuracy is about 90%. In some embodiments, the accuracy is about 87%. In some embodiments, the accuracy is about 86%. In some embodiments, the accuracy is about 80%. In some embodiments, the accuracy is about 70%. In some embodiments, the accuracy is about 60%.

The specificity of the methods of diagnosis and/or prognosis can be a measure of the proportion of subjects that are actually negative for a condition which are correctly identified as being negative for the condition by the model. The specificity of a model can be equal to the number of true negatives divided by the sum of the number of true negatives and false positives. In other words, the specificity of a model can be the probability of a negative test result given that the subject is actually negative for the condition. In some embodiments, the specificity of the methods described herein is the number of subjects without rejection that are predicted by the methods described herein to not have rejection divided by the total number of subjects predicted to not have rejection using the methods described herein. In some embodiments, the comparing step of the methods described herein comprises assessing (e.g., predicting, diagnosing, identifying, etc.) a rejection response with a specificity of about 60-100%. In some embodiments, the specificity is about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, but no more than 100%. In some embodiments, the specificity is about 60-65%, 65-70%, 70-75%, 75-80%, 80-85%, 85-90%, 90-95%, or 95-100%, but no more than 100%. In some embodiments, the specificity is about 90%. In some embodiments, the specificity is about 80%. In some embodiments, the specificity is about 70%. In some embodiments, the specificity is about 66.67%. In some embodiments, the specificity is about 60%.

The sensitivity of the methods of diagnosis and/or prognosis can be a measure of the proportion of subjects that are actually positive for a condition which are correctly identified as being positive for the condition by the model. The sensitivity of a model can be equal to the number of true positives divided by the sum of the number of true positives and false negatives. In other words, the sensitivity of a model can be the probability of a positive test result given that the subject is actually positive for the condition. In some embodiments, the sensitivity of the methods herein is the number of subjects with rejection that are predicted by the methods described herein to have rejection divided by the total number of subjects predicted to have rejection using the methods described herein. In some embodiments, the comparing step of the methods described herein comprises assessing (e.g., predicting, diagnosing, identifying, etc.) a rejection response with a sensitivity of about 70-100%. In some embodiments, the sensitivity is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, but no more than 100%. In some embodiments, the sensitivity is about 70-75%, 75-80%, 80-85%, 85-90%, 90-95%, or 95-100%, but no more than 100%. In some embodiments, the sensitivity is about 70%. In some embodiments, the sensitivity is about 92%. In some embodiments, the sensitivity is about 99%.

The present invention further encompasses methods of treating a subject in need of treatment for a transplant rejection response after receiving a heart or kidney transplant. In some embodiment, the number of the non-H LA antibodies disclosed herein with altered level in a sample obtained from a subject, e.g., increased or present compared to reference levels, can inform the method of treatment. In some embodiments, the detection of increased level of one or more of the non-HLA antibodies described herein compared to reference levels indicates that the subject needs treatment.

In certain embodiments, if one or more of the non-HLA antibody disclosed herein is detected in a biological sample, standard treatment methods for removal of the antibody may be employed. For example, the attending clinician administer, perform or request plasmapheresis to be conducted on the subject. Plasmapheresis is a well-known procedure that can selectively remove harmful antibodies from a subject's circulation. In certain embodiments, the method comprises contacting a biological sample obtained from the subject with the composition disclosed herein, measuring levels of the one or more antibodies in the sample, and administering a treatment for transplant rejection to the subject when there are increased levels of the one or more antibodies disclosed herein, compared to reference levels of the one or more antibodies.

In certain embodiments, the treatment protocols for AMR use permutations of a multiple-prong approach that include, but are not limited to: (1) the suppression of the T-cell dependent antibody response, (2) the removal of reactive antibody, (3) the blockade of the residual alloantibody, and (4) the depletion of naive and memory B-cells. In certain embodiments, the treatment regimen is application of plasmapheresis. In certain embodiments, the treatment regimen is administration of rituximab. In certain embodiments, the treatment regimen includes administration of at least one proteasome inhibitor-based therapy, such as but not limited to bortezomib. In certain embodiments, the treatment regimen is administration of mycophenolate mofetil. Additional treatment methods are known in the art, e.g., see Levine MH, et al., Treatment options and strategies for antibody mediated rejection after renal transplantation. Semin Immunol. 2012 April; 24(2):136-42, which is incorporated by reference.

In certain embodiments, the therapeutic agents are selected from tacrolimus, mycophenolate mofetil, and Everolimus, with or without corticosteroids. In certain embodiments, the therapeutic agents are tacrolimus, mycophenolate mofetil, and corticosteroids. In certain embodiments, the therapeutic agents are tacrolimus, Everolimus, and corticosteroids.

In certain embodiments, the treatment further includes a steroid. In some embodiments, the steroids include, but are not limited to, corticosteroids (e.g., glucocorticoids and mineralocorticoid). In some embodiments, the corticosteroid is selected from prednisone (Deltasone, Orasone), budesonide (Entocort EC), and prednisolone (Millipred). In some embodiments, steroids are used to decrease inflammation and reduce the activity of the immune system. In certain embodiments, the treatment does not include a steroid.

In certain embodiments, the method comprises administering a therapeutically effective amount of one or more of therapeutic agents to the subject. In some embodiments, the therapeutic agent is a Janus kinase inhibitors (e.g., tofacitinib (Xeljanz)). In some embodiments, the therapeutic agent is a Calcineurin inhibitor (e.g., cyclosporine (Neoral, Sandimmune, SangCya), or tacrolimus (Astagraf XL, Envarsus XR, Prograf)). In some embodiments, the therapeutic agent is an mTOR inhibitor (e.g., sirolimus (Rapamune), or everolimus (Afinitor, Zortress)). In some embodiments, the therapeutic agent is an IMDH inhibitor (e.g., azathioprine (Azasan, Imuran), leflunomide (Arava), or mycophenolate (CellCept, Myfortic)). In some embodiments, the therapeutic agent is a biologics (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio)) or belatacept (Nulojix). In some embodiments, the therapeutic agent is a monoclonal antibody (e.g., basiliximab (Simulect) or daclizumab (Zinbryta)). In certain embodiments, the treatment includes one or more therapeutic agents selected from the above.

In certain embodiments, the treatment further includes a steroid. In some embodiments, the steroids include, without being limited to, corticosteroids (e.g., glucocorticoids and mineralocorticoid). In some embodiments, the corticosteroid is selected from prednisone (Deltasone, Orasone), budesonide (Entocort EC), and prednisolone (Millipred). In some embodiments, steroids are used to decrease inflammation and reduce the activity of the immune system. In certain embodiments, the treatment does not include a steroid.

In certain embodiments, the therapeutic agents are selected from tacrolimus, mycophenolate mofetil, and Everolimus, with or without corticosteroids. In certain embodiments, the therapeutic agents are tacrolimus, mycophenolate mofetil, and corticosteroids. In certain embodiments, the therapeutic agents are tacrolimus, Everolimus, and corticosteroids.

In another embodiment, the present invention provides a kit. Such a kit is a packaged combination including the basic elements of: (a) a composition comprising a collection of solid-phase substrates coated with one or more homogenous populations of binding agents, wherein each homogenous population of binding agents specifically binds to an antibody that is directed against a single antigen selected from the group consisting of (DEXI), C—X—C motif chemokine ligand 11 (CXCL11), cytokeratin 18 (KRT18), cytokeratin 8 (KRT8), Tubulin, including tubulin alpha 1 b (also referred to as TUBα1b or TUBA1B), latrophilin 1 (LPHN1), Colony stimulating factor 2 (CSF2), Signal Transducer And Activator Of Transcription 6 (STATE), lectin galactoside-binding soluble 3 (LGALS3), SHC Adaptor Protein 3 (SHC3), Glyceraldehyde-3-phosphate dehydrogenase (GAPDH), Glutathione 5-Transferase theta-1 (GSTT1), phospholipase A2 receptor 1 (PLA2R1), Interleukin 8 (IL-8), lectin galactoside-binding soluble 8 (LGALS8), Small Nuclear Ribonucleoprotein Polypeptide N (SNPRN), Myosin, Peroxisomal trans-2-enoyl-CoA Reductase (PECR), vimentin (VIM), ATP synthase H+transporting mitochondrial F1 complex beta polypeptide (ATP5B), Collagen II, Prelamin-A/C (LMNA), small nuclear ribonucleoprotein polypeptide B (SNRPB2), fibronectin 1 (FN1) and Vinculin (VCL) and (b) reagents for detecting the binding of the one or more homogenous populations of binding agents to the antibodies.

In another embodiments, each homogenous population of binding agents specifically binds to an antibody that is directed against a single antigen selected from the group consisting of Tubulin, LPHN1, SNRPN, KRT18, KRT8, LGALS3, SNRPB2, DEXI, Collagen II, PLA2R1, GSTT1, VCL, CSF2, LGALS8, STAT6, GAPDH, IL-8, SHC3, ENO1, AGRN, EMCN, SSB, TG, PECR, NPHS1, FN1, Myosin, VIM, ATP5B, LMNA, CXCL11, Actin, FLT3LG, PRKCH, and IL-21. In certain embodiments, the kits also include instructions for use thereof.

In a further embodiment, the kit can comprise one or more reference samples. Reference samples can be any biological samples as used herein. In another embodiment, the kit can comprise one or more reference levels of the non-HLA antibodies disclosed herein.

All patents and publications mentioned in this specification are indicative of the level of those skilled in the art to which the invention pertains. All patents and publications cited herein are incorporated by reference to the same extent as if each individual publication was specifically and individually indicated as having been incorporated by reference in its entirety.

### Exemplary Embodiments

The following are examples of embodiments described herein:

Embodiment 1: A composition comprising a collection of solid-phase substrates coated with one or more homogenous populations of binding agents, wherein each homogenous population of binding agents specifically binds to an antibody that is directed against a single antigen selected from the group consisting of: dexamethasone-induced transcript (DEXI), C—X—C motif chemokine ligand 11 (CXCL11), cytokeratin 18 (KRT18), cytokeratin 8 (KRT8), Tubulin, including tubulin alpha 1 b (also referred to as TUBα1b or TUBA1B), latrophilin 1 (LPHN1), Colony stimulating factor 2 (CSF2), Signal Transducer And Activator Of Transcription 6 (STAT6), lectin galactoside-binding soluble 3 (LGALS3), SHC Adaptor Protein 3 (SHC3), Glyceraldehyde-3-phosphate dehydrogenase (GAPDH), Glutathione S-Transferase theta-1 (GSTT1), phospholipase A2 receptor 1 (PLA2R1), Interleukin 8 (IL-8), lectin galactoside-binding soluble 8 (LGALS8), Small Nuclear Ribonucleoprotein Polypeptide N (SNPRN), Myosin, Peroxisomal trans-2-enoyl-CoA Reductase (PECR), vimentin (VIM), ATP synthase H+transporting mitochondrial F1 complex beta polypeptide (ATP5B), Collagen II, Prelamin-A/C (LMNA), small nuclear ribonucleoprotein polypeptide B (SNRPB2), fibronectin 1 (FN1), nephrosis 1, congenital, Finnish type (NPHS1), Thyroglobulin (TG), and Vinculin (VCL).

Embodiment 2: The composition of embodiment 1, wherein the collection of solid-phase substrates further comprises one or more additional homogenous populations of binding agents, wherein each additional homogenous population of binding agents specifically binds to an antibody that is directed against a single additional antigen selected from the group consisting of: Alpha-enolase (ENO1), Agrin (AGRN), Endomucin (EMCN), Sjogren syndrome antigen B (SSB), Actin, fms-related tyrosine kinase 3 ligand (FLT3LG), Protein kinase C eta (PRKCH), and Interleukin 21 (IL-21).

Embodiment 3: The composition of embodiment 1 or 2, wherein the solid-phase substrates is porous or non-porous.

Embodiment 4: The composition of embodiment 2 or 3, wherein the solid-phase substrates comprise particles, nanoparticles, beads, nanobeads or microspheres.

Embodiment 5: The composition of 4, wherein the beads are polystyrene beads.

Embodiment 6: The composition of any one of the preceding embodiments, wherein the collection of solid-phase substrates comprises a microarray.

Embodiment 7: The composition of any one of the preceding embodiments, wherein the solid-phase substrates are fluorescently labeled, magnetically labeled, or radio labeled.

Embodiment 8: The composition of any one of the preceding embodiments, wherein the solid-phase substrates are labeled with a small molecule.

Embodiment 9: The composition of any one of the preceding embodiments, wherein the one or more homogenous populations of binding agents are conjugated to the surface of the solid-phase substrates.

Embodiment 10: The composition of embodiment 9, wherein the conjugation is covalent.

Embodiment 11: The composition of any one of the preceding embodiments, wherein the one or more homogenous populations of binding agents are attached to the surface of the solid-phase substrates by affinity.

Embodiment 12: The composition of any one of the preceding embodiments, wherein the binding agent is a polypeptide.

Embodiment 13: The composition of any one of the preceding embodiments, wherein the solid-phase substrates are coated with at least three different homogenous populations of binding agents that bind to at least three different antigens.

Embodiment 14: The composition of embodiment 13, wherein the substrates are coated with multiple different homogenous populations of binding agents that bind to the antibodies consisting of antibodies to Tubulin, LPHN1, SNRPN, KRT18, KRT8, LGALS3, SNRPB2, DEXI, Collagen II, PLA2R1, GSTT1, VCL, CSF2, LGALS8, and STAT6.

Embodiment 15: The composition of embodiment 13, wherein the substrates are coated with multiple different homogenous populations of binding agents that bind to the antibodies consisting of antibodies to Tubulin, LPHN1, TG, GAPDH, FN1, NPHS1, VIM, Myosin, VCL and PECR.

Embodiment 16: The composition of embodiment 15, wherein the substrates are further coated with multiple different homogenous populations of binding agents that bind to the antibodies consisting of antibodies to ENO1, AGRN, EMCN, SSB, Actin, FLT3LG, PRKCH, and IL-21.

Embodiment 17: The composition of embodiment 13, wherein the substrates are coated with multiple different homogenous populations of binding agents that bind to the antibodies consisting of antibodies to DEXI, LGALS3, SNPRN, CSF2, IL-8, STAT6, LGALS8, KRT18, KRT8, GSTT1, LMNA, Collagen II, ATP5B, SNRPB2 and PLA2R1.

Embodiment 18: The composition of embodiment 13, wherein the substrates are coated with multiple different homogenous populations of binding agents that bind to the antibodies consisting of antibodies to Tubulin, SHC3 and CXCL11.

Embodiment 19: The composition of embodiment 13, wherein the substrates are coated with multiple different homogenous populations of binding agents that bind to the antibodies consisting of antibodies to DEXI, EMCN, SNRPN, LPHN1 and SSB.

Embodiment 20: The composition of embodiment 13, wherein the substrates are coated with multiple different homogenous populations of binding agents that bind to the antibodies consisting of antibodies to KRT18, GAPDH, AGRN, ENO1 and EMCN.

Embodiment 21: The composition of embodiment 13, wherein the substrates are coated with multiple different homogenous populations of binding agents that bind to the antibodies consisting of antibodies to Tubulin, LPHN1, SNRPN, KRT18, KRT8, LGALS3, SNRPB2, DEXI, Collagen II, GAPDH, ENO1, AGRN, EMCN, SSB, PLA2R1, GSTT1, VCL, CSF2, LGALS8, STAT6, IL-8, and SHC3.

Embodiment 22: The composition of embodiment 1, wherein the single antigen is selected from the group consisting of: LPHN1, TG, DEXI, CSF2, IL-8, LGALS3, SNPRN, STATE, SHC3, and LGALS8.

Embodiment 23: The composition of embodiment 2, wherein the single additional antigen is selected from the group consisting of: EMCN and SSB.

Embodiment 24: The composition of any of the preceding embodiments, wherein at least one solid-phase substrate is detectably distinguishable from at least one other solid-phase substrate.

Embodiment 25: A method for determining the presence of one or more antibodies in a biological sample obtained from a subject, the method comprising: contacting the biological sample with the composition of any of embodiments 1-24, and detecting the binding of the one or more homogenous populations of binding agents to the one or more antibodies.

Embodiment 26: The method of embodiment 25, wherein the subject is a mammal.

Embodiment 27: The method of embodiment 26, wherein the subject is a human.

Embodiment 28: The method of any of embodiments 25-27, wherein the subject has received or will receive a heart or kidney transplant.

Embodiment 29: The method of embodiment 28, wherein the heart transplant is an allograft heart or kidney transplant.

Embodiment 30: The method of any of embodiments 25-29, wherein the biological sample is blood, plasma, serum, urine, spinal fluid, lymph fluid, synovial fluid, cerebrospinal fluid, tears, saliva, milk, mucosal secretion, effusion, sweat, biopsy aspirates, ascites or fluidic extracts.

Embodiment 31: The method of any of embodiments 25-30, wherein the detecting is by measuring a fluorescence intensity or by an immunological analysis.

Embodiment 32: A method for diagnosing a transplant rejection response in a subject that has undergone a heart or kidney transplant, the method comprising: contacting a biological sample obtained from the subject with the composition of any of embodiments 1-24, and measuring the levels of the one of more antibodies in the sample; wherein increased levels of the one or more antibodies, compared to reference levels, indicates that the subject has developed a transplant rejection response in response to the heart or kidney transplant.

Embodiment 33: A method for predicting the likelihood of a transplant rejection response in a subject in need of a heart or kidney transplant, the method comprising: contacting a biological sample from the subject with the composition of any of embodiments 1-24, and measuring the levels of the one or more antibodies in the sample; wherein increased levels of the one or more antibodies, compared to reference levels, indicates that the subject has an increased likelihood of developing a transplant rejection response after a heart or kidney transplant.

Embodiment 34: A method of treating a subject in need of treatment for a transplant rejection response after receiving a heart or kidney transplant, the method comprising: contacting a biological sample obtained from the subject with the composition of any of embodiments 1-24, measuring levels of the one or more antibodies in the sample, and administering a treatment for transplant rejection to the subject when there are increased levels of the one or more antibodies, compared to reference levels of the one or more antibodies.

Embodiment 35: A kit comprising: the composition of any of embodiments 1-24, and reagents for detecting the binding of the one or more homogenous populations of binding agents to the antibodies.

Embodiment 36: The kit of embodiment 35, further comprising one or more reference samples.

### Examples

The following examples are provided for illustrative purposes. These are intended to show certain aspects and embodiments of the present invention but are not intended to limit the invention in any manner.

Example 1. Non-HLA Antibodies Associated with Cardiac and Renal Allograft Rejection

There is a growing body of evidence that the development of post-transplant antibodies against non-HLA antigens is associated with rejection and decreased long-term graft survival. This Example describes the use of sera from renal and heart transplant recipients obtained before and after transplantation to create a panel of non-HLA antigenic targets that can be used to identify transplant recipients with circulating non-HLA antibodies that may portend risk of graft injury and loss.

Non-HLA antibodies reactive with endothelial cells were identified by testing sera from cardiac and renal transplant recipients diagnosed with acute rejection in the absence of detectable circulating donor-specific HLA antibodies, for binding in a flow crossmatch to human primary arterial endothelial cells (Zhang and Reed, Transplantation 2005). Sera positive in the endothelial cell crossmatch were tested neat and/or following absorption and elution from aortic endothelial cells on protoarrays containing >9000 full-length human protein antigens. Non-HLA antibody targets associated with EC plasma membrane and autoantigens were classified using a bioinformatics and gene ontological approach. These studies resulted in the identification of 31 non-HLA antigenic targets (Table 1). These and other non-HLA antigens were conjugated to polystyrene beads to develop a multiplex bead array for further high throughput testing on cardiac, and pediatric and adult renal transplant recipients (Table 1).

TABLE 1
Non-HLA Antibodies Associated with Cardiac and Renal Allograft
Rejection

| # | Marker ID | Antigen | Gene Name |
|---|---|---|---|
| 1 | Cardiac 1 | DEXI | dexamethasone-induced transcript |
| 2 | Cardiac 2 | EMCN | Endomucin |
| 3 | Cardiac 3 | SNRPN | Small Nuclear Ribonucleoprotein Poly- |
| | | | peptide N (smith antigen core sequence) |
| 4 | Cardiac 4 | NTRK1 | Neurotrophic Tyrosine Kinase, Receptor, |
| | | | Type 1 |
| 5 | Cardiac 5 | ROR1 | Receptor Tyrosine Kinase-Like Orphan |
| | | | Receptor 1 |
| 6 | Cardiac 6 | LGALS3 | lectin, galactoside-binding, soluble, 3 |
| 7 | Cardiac 7 | ANXA2R | annexin A2 receptor |
| 8 | Cardiac 8 | IYD | iodotyrosine deiodinase |
| 9 | Cardiac 9 | EMCN | Endomucin |
| 10 | Cardiac 10 | FGFR1 | fibroblast growth factor receptor 1 |
| 11 | Cardiac 11 | HARS | Jo-1 |
| 12 | Cardiac 12 | SPN | Sialophorin, CD43 |
| 13 | Cardiac 13 | SSB | Sjogren syndrome antigen B (autoantigen |
| | | | La) |
| 14 | Cardiac 14 | IL-8 | interleukin 8, CXCL8 |
| 15 | Cardiac 15 | CCP | cyclic citrullinated peptide |
| 16 | Cardiac 16 | VEGFA | vascular endothelial growth factor A |
| 17 | Cardiac 17 | DQA1*01:02 | HLA-DQA1 |
| 18 | Cardiac 18 | LPHN1 | latrophilin 1 |
| 19 | Cardiac 19 | TG | Thyroglobulin |
| 20 | Renal 1 | FGF2 | fibroblast growth factor receptor 2 |
| 21 | Renal 2 | CSF2 | Colony stimulating factor 2 |
| 22 | Renal 3 | NGF | Nerve growth factor |
| 23 | Renal 4 | LGALS8 | lectin, galactoside-binding, soluble, 8 |
| 24 | Renal 5 | LGALS1 | lectin, galactoside-binding, soluble, 1 |
| 25 | Renal 6 | MAPK1 | Mitogen-Activated Protein Kinase 1 |
| 26 | Renal 7 | SDF1B | C-X-C Motif Chemokine Ligand 12 |
| 27 | Renal 8 | VWF | Von Willebrand Factor |
| 28 | Renal 9 | STAT6 | Signal Transducer And Activator Of |
| | | | Transcription 6 |
| 29 | Renal 10 | SHC3 | SHC Adaptor Protein 3 |
| 30 | Renal 11 | GNG5 | G Protein Subunit Gamma 5 |
| 31 | Renal 12 | IL18R1 | Interleukin 18 Receptor |

Cardiac transplant rejection discovery cohort. The cardiac transplant discovery cohort was identified from 12 cardiac allograft recipients transplanted at UCLA between 2001-2005 who tested positive for endothelial cell (EC) antibodies by EC flow crossmatch (ECXM) and were negative for HLA DSA and MICA antibodies (Zhang Transplantation 2005). Six ECXM-, HLA DSA-patients without rejection were included as controls. Patients were typed for HLA by LABType SSO DNA typing (One Lambda, Canoga Park, CA) according to the manufacturers' specifications. MICA antibodies and HLA class I and class II antibodies were identified using a Single Antigen Luminex assay (One Lambda, Canoga Park, CA). Acute cellular rejection (ACR) and AMR were diagnosed by endomyocardial biopsy (EMB) according to the International Society for Heart and Lung Transplantation (ISHLT) criteria and as reported previously (Zhang Transplantation 2005, Stewart J Heart and Lung Transplant, 005). The mean time for collection of sera samples from day of transplant was 67 days and from day of the rejection+biopsy was 14 days.

Renal transplant rejection discovery cohort. The renal transplant discovery cohort (n=16) was identified from renal allograft recipients transplanted at UCLA between 2010-2015 who were diagnosed with rejection, and tested positive for endothelial cell antibodies by ECXM (Zhang Transplantation 2005). Pre-transplant serum from 3 of the 16 patients served as a negative control. The patient's sera were tested both neat and following adsorption and elution from endothelial cells. The mean time for collection of sera samples from day of transplant was 21 days and from day of rejection+biopsy was 23 days.

Single center cardiac transplant validation cohort. The single center cohort consisted of 63 heart transplant recipients transplanted at UCLA between 2009-2016. Sera were collected at the time of EMB. Rejection (n=42, ACR >1R) was scored according to the ISHLT revision of the 1990 working formulation for heart transplant rejection. The median and interquartile range in days from sera collection to EMB for both rejection and no rejection samples was 0 (p<0.26). Post-transplant, patients were maintained on triple-drug immunosuppression (tacrolimus, mycophenolate mofetil, and corticosteroids).

Pediatric renal transplant validation cohort. Eighty-three pediatric kidney transplant patients transplanted at UCLA were enrolled in this study from August 2005 to November 2014. Twenty-one patients were excluded from this analysis due to >1 study sample was missing at the listed time points. The remaining 62 patients were included in this retrospective study. This study was approved by the UCLA Institutional Review Board (#002375) and conforms with the 1964 Declaration of Helsinki and its later amendments or comparable ethical standards and the principles of the Declaration of Istanbul. Informed consent from legal guardians was obtained for all patients. Immunosuppressive regimens included induction with either ATG for a PRA ≥30%, delayed graft function, or rapid steroid withdrawal protocol or anti-CD25 monoclonal antibody for those with a PRA <30%, Maintenance immunosuppression consisted of steroid-free or steroid-based immunosuppression, a calcineurin inhibitor, and an anti-metabolite. Acute rejection and chronic rejection were treated with previously described protocols (Pearl, Pediatr Nephrol, 2016), Patients underwent protocol biopsies at 6, 12, and 24 months post-transplantation or for clinical indication. Biopsy samples were evaluated based on the 2013 Banff Criteria (Haas, Am J Transplant, 2014). Blood samples were obtained pre-transplantation and at 6, 12, and 24 months post-transplantation and during episodes of kidney transplant rejection.

Adult renal transplant validation cohort. The cohort consisted of 163 renal transplant recipients transplanted at UCLA between 2006-2012, Biopsy samples were evaluated based on the 2013 Banff Criteria (Haas, Am J Transplant, 2014), Sera were collected at the time of biopsy (+1-6 weeks). Post-transplant, patients were maintained on triple-drug immunosuppression (tacrolimus, mycophenolate mofetil, and corticosteroids). induction was primarily solumedrol and basiliximab or anti-thymocyte globulin. IVIG is used to augment immunosuppression at the time of transplant for patients with DSA that is identified within one year of transplant (current). For patients with historic DSA, the use of IVIG at the time of transplant is at the discretion of the attending nephrologist. This study was approved by the UCLA Institutional Review Board (#16-000786).

HLA typing and HLA DSA testing. All patients were typed for HLA by LABType SSO DNA typing (One Lambda, Canoga Park, CA) according to the manufacturers' specifications, MICA antibodies and HLA class I and class II antibodies were identified using a Single Antigen Luminex assay (One Lambda, Canoga Park, CA).

Renal Transplant Rejection. ACR and AMR were diagnosed by renal biopsy according to the Banff criteria (Haas M et al, AJT 2014).

Protoarray Assay for discovery of non-HLA antigens. Serum antibodies were profiled with a human protein microarray as previously described. Briefly, the InVitrogen Human ProtoArray v5.0 containing over 9,000 proteins from a baculovirus-based expression system was blocked with blocking buffer for 1 h and then incubated with 5 μL of serum diluted in PBST buffer at 1:150 dilution for 90 min. The slides were washed with 5 mL of fresh PBST buffer, 4 times for 10 min each, and probed with goat anti-human Alexa fluor 647 IgG secondary antibody (Molecular Probes, Eugene, Oreg.) for 90 min. After a second wash with PBST buffer, the slides were dried and scanned using a GenePix 4100A fluorescence microarray scanner and GenePix Pro 6.0 software (Molecular devices, Sunnyvale, Calif.). Protein arrays included 12 rejection+ECXM+sera, and 6 rejection− ECXM− sera to serve as technical controls. Array normalization and analysis was performed using Prospector 2.0 software (Life Technologies, Grand Island, N.Y.) as previously described. Gene ontological analysis of the 366 antigens was assessed by DAVID gene ontology analysis software (available on the world wide web at david.ncifcrf.gov) to identify enriched biological themes as previously described.

Development of the Multiplex Non-HLA Antigen Panel for detection of non-HLA antibodies. The non-HLA antigen targets newly described herein were discovered using the selected discovery cohorts described above and in Table 1. Gene ontological analyses and tissue expression data were used to select the most biologically relevant non-HLA targets to be included in the non-HLA panel. Non-HLA multiplex bead panel of single antigen beads were manufactured by, and reagents for use to screen sera from selected cohorts of cardiac, adult renal and pediatric renal transplant recipients were provided by, Immucor, Inc. (Peachtree Corners, GA). Briefly, 40 μL of antigen-coated beads were incubated with 10 μL of patient's serum for 30 min. Unbound serum was removed by washing, and the beads were stained with 50 μL of conjugate containing phycoerythrin (PE) conjugated goat anti-human IgG diluted 1:10 in wash buffer and incubated in the dark on a shaking platform for 30 min. Results were examined by reading the median fluorescence intensity (MFI) of IgG binding on the Luminex 100 analyzer (Luminex, Austin, Tex.). To determine a positive threshold for the non-HLA antibody luminex assay, non-HLA antibodies were assessed in sera isolated from 44 healthy individuals. The median MFI of antibody reactivity to the 18 antigens significantly associated with cardiac allograft rejection was 510 MFI (range: 67-8367 MFI, SD=900). A positive threshold of MFI >1000 was chosen as per our prior experience with luminex-based solid phase antibody detection methods.

Statistics. The odds ratio associating non-H LA antibodies with allograft rejection were determined to be significantly greater than 1 by 2-sided Fisher's exact test (p<0.1; StataCorp. 2015. Stata Statistical Software: Release 14. College Station, Tex.: StataCorp LP). The p<0.1 threshold for significance was set following standard practice for discovery analysis (Fan, L. et al. Am J Transplant 2011) and default alpha-value (p value) of 0.1 in STATA to prevent exclusion of antibodies that may interact with each other. Confidence intervals (95%) were constructed assuming the estimates of standard error are asymptotically independent and normal.

In the cluster analysis, ordering of the non-HLA markers, circle size and illustration of the inner boxes identifying clusters is according to the numeric value of the pairwise correlation coefficient. The analysis was performed using hierarchal clustering with complete linkage and Euclidean distance in the R Corrplot application (Simko, T. w. a. V. 2017).

The rpart function in the R library (the R software package version 3.4.0 (available on the world wide web at www.r-project.org/)) was applied to conduct the classification and regression tree (CART) analysis. In order to avoid over-fitting and to select a parsimonious set of predictor variables, the maxdepth (the maximum depth of any node of the final tree, with the root node counted as depth 0) was set to 3, and the minsplit (the minimum number of observations that must exist in a node, in order for a split to be attempted) was set to 5. All other computer software parameters were set to their default values.

Cardiac transplant non-HLA panel development: We hypothesized that antibodies targeting non-HLA antigens expressed by the allograft endothelium would be identified through protein microarray analysis using sera from cardiac allograft recipients with biopsy proven rejection. Sera from twelve cardiac transplant recipients without HLA or MICA DSA, but with biopsy proven rejection and with positive reactivity in the ECXM (median of 117 MCS; range: 62-529 MCS) were assembled as a discovery cohort. The discovery sera samples were collected an average 14 days from the EMB (range: 0-76 days). EMB were obtained an average 67 days after transplant (range: 20-222 days). An additional six sera that were not associated with rejection and that were ECXM negative and MICA DSA negative were used as controls. The 18 sera (12 rejection+/ECXM+ and 6 rejection-/ECXM-controls) were hybridized to protein microarrays containing 9,000 full-length human proteins. Bioinformatic analysis of the protein microarrays identified 366 rejection-associated antigens with significantly increased fluorescence intensity (>1.5 fold; p<0.05) indicating positive antibody reactivity in sera isolated from rejection+/ECXM+patients compared to rejection-/ECXM-controls. Next, gene ontological analysis of the 366 non-HLA antigens was performed to identify those that were most biologically relevant with respect to known functional characteristics. From this analysis, twenty-two plasma membrane antigens and 10 known autoantigens were selected as these are likely target antigens expressed on the endothelial cell surface and may contribute to a positive ECXM. Of these, 19 were able to be expressed and conjugated to luminex polystyrene beads for further downstream high throughput testing (Table 1). Other non-HLA antigens (9 cardiac, 30 renal, 8 lung, and 1 liver) (Table 4) and were selected to generate a multiplex panel of 67 non-HLA antigens.

Renal transplant non-HLA panel development: We tested sera from 16 renal transplant recipients without HLA or MICA DSA, but with biopsy proven rejection and a positive ECXM. An additional 3 sera collected pre-transplant from 3/16 recipients were used as a negative control. Sera were tested in parallel neat and following adsorption and elution on endothelial cells. The non-adsorbed and eluted sera were tested on protein microarrays which resulted in the identification of 1252 antigenic targets (>1.5 fold increase, p<0.05) compared to pre-transplant sera. Prospector analysis identified antibodies present in both the neat and eluted rejection sera reacting with 386 distinct proteins. Of these, 251 were excluded from the analysis as they were present in pre-transplant sera. This resulted in 135 unique proteins as potential candidates for constructing a non-HLA panel. After gene ontology and frequency analysis, 12/135 antigenic targets were selected based on their expression profile and functional characteristics and added to the 67 antigens used in the cardiac multiplex bead array to generate the Renal Mutliplex Bead Array.

Identification of non-HLA antibodies associated with allograft rejection. The multiplex bead array was used to screen sera samples from 34 no rejection and 33 rejection cardiac transplant samples from patients transplanted at UCLA between 2009-2016 to determine if they developed non-HLA antibodies. A higher percentage of cardiac transplant recipient samples with rejection were observed with 10 non-HLA antibodies (Tubulin, LPHN1, Thyroglobulin (TG), GAPDH, FN1, NPHS1, VIM, Myosin, VCL, PECR) compared to non-rejection patient sera samples (Table 2). LPHN1 and Thyroglobulin (TG) are newly described herein.

TABLE 2
Non-HLA Abs Associated with Rejection in the UCLA Adult
Cardiac Cohort

| | No Rejection | Rejection | Fisher-exact |
|---|---|---|---|
| Non-HLA Ab | (n = 34) | (n = 33) | p-value |
| Tubulin | 23.53% | 36.36% | 0.189 |
| LPHN1 | 17.65% | 33.33% | 0.116 |
| TG | 5.88% | 12.12% | 0.322 |
| GAPDH | 0 | 9.09% | 0.114 |
| FN1 | 0 | 9.09% | 0.114 |
| NPHS1 | 2.94% | 12.12% | 0.169 |
| VIM | 2.94% | 9.09% | 0.295 |
| Myosin | 5.88% | 12.12% | 0.322 |
| VCL | 5.88% | 15.15% | 0.201 |
| PECR | 5.88% | 15.15% | 0.201 |

Identification of non-HLA antibodies associated with renal allograft rejection. The multiplex bead array was used to screen sera samples isolated from recipients of pediatric (samples: n=95 no rejection and n=34 rejection) and adult (samples: n=90 no rejection and n=70 rejection) renal transplants. Patients in the rejection group experienced at least one histologically proven rejection episode during the study period. The pediatric and adult renal sample cohorts were analyzed independently (FIGS. 1A and B). From these independent analyses 15 and 3 antibodies to non-HLA antigens were identified as significantly associated with rejection, respectively (p<0.01, and a risk ratio >1) (FIG. 1). In the pediatric cohort, antibodies to 15 non-HLA antigens (y-axis) were identified to be significantly associated with the time to first renal rejection with an odds ratio >1 (x-axis) in pediatric renal transplant recipients (FIG. 1A). Seven of these, DEXI, CSF2, IL-8, LGALS3, SNPRN, STATE and LGALS8, are newly described herein as significantly related to renal transplant rejection. Bars represent the 95% confidence interval (CI). Antibodies to 3 additional non-HLA antigens (Tubulin, CXCL11, SHC3) were significantly associated with renal allograft rejection in adult renal transplant recipients (FIG. 1B). SHC3 is newly identified herein as significantly related to renal transplant rejection.

Table 3 shows a list of non-HLA antibodies identified as associated with rejection after adult cardiac transplant (first column), Pediatric renal (second column) and Adult renal transplant (third column). These same targets are listed in FIGS. 1-3, 5 and 6, with indicators of those which are predictive in CART analysis, sort independently, are newly described, and cluster together in the correlation matrix.

TABLE 3
Non-HLA Abs Associated with Rejection in the UCLA
Adult Cardiac Cohort, UCLA Pediatric Renal, and UCLA
Adult Renal cohorts.

| Cardiac, Adult | Renal, pediatric | Renal, adult |
|---|---|---|
| Tubulin | DEXI | Tubulin |
| LPHN1 | LGALS3 | SHC3 |
| TG | SNPRN | CXCL11 |
| GAPDH | CSF2 | |
| FN1 | IL-8 | |
| NPHS1 | STAT6 | |
| VIM | LGALS8 | |
| Myosin | KRT18 | |
| VCL | KRT8 | |
| PECR | GSTT1 | |
| | LMNA | |
| | Collagen II | |
| | ATP5B | |
| | SNRPB2 | |
| | PLA2R1 | |

A correlation matrix analysis of non-HLA antibodies associated with rejection after adult cardiac transplant was performed (FIG. 2A). Non-HLA antibodies associated with adult cardiac allograft rejection selectively cluster into 4 groups with one of them clustering independently (VCL). (Non-HLA antibodies associated with pediatric renal allograft rejection selectively cluster into 6 groups (FIG. 2B) with four of them clustering independently (PLAR1, CSF2, GSTT1, and LGALS8). Seven of the non-HLA antibodies are newly identified to be associated with renal allograft rejection in this study (CSF2, SNPRN, LGALS8, STAT6, IL-8, DEXI, and LGALS3). The non-HLA antibodies found to be significantly associated with transplant rejection in the pediatric renal cohort were applied to the adult renal transplant cohort to similarly assess for correlation (FIG. 2C). Antigens that are independently correlated during a rejection episode are similar between the pediatric and adult renal transplant patients with rejection (adult renal: CSF2, GSTT1).

The pediatric and adult cohorts were then combined into one analysis to determine how the antibodies specific for the panel of 18 non-HLA targets independently sort in rejection (FIG. 3, n=104). In rejection samples, the antibodies cluster into 9 groups, with four of the 18 clustering independently (PLA2R, STAT6, GSTT1, and CSF2). Three of these four (STAT6, GSTT1, and CSF2) are newly identified herein though the proteomics based discovery.

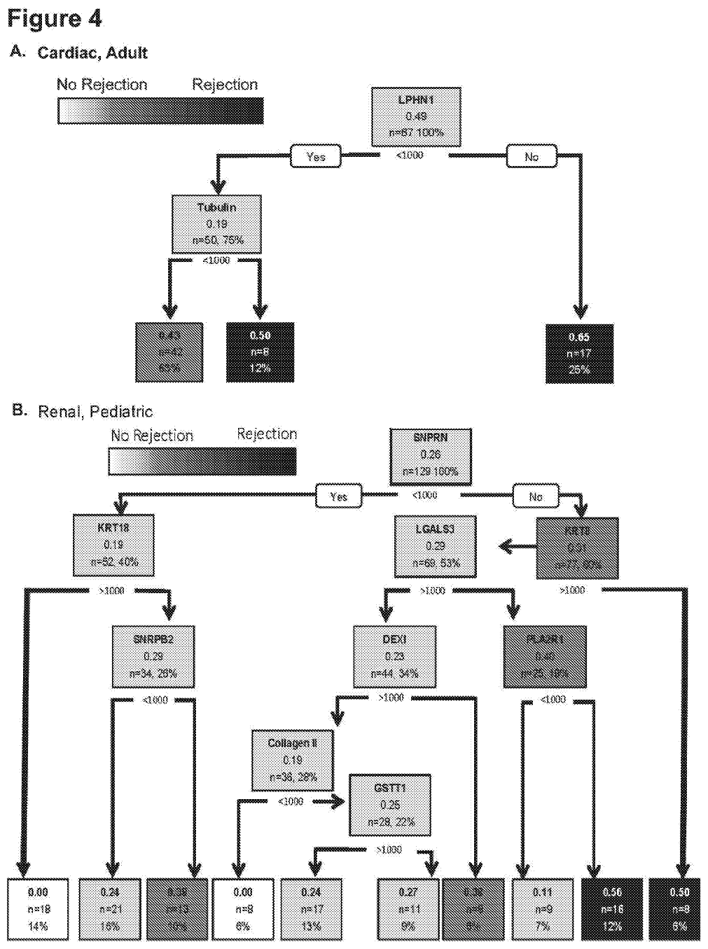

A classification and regression tree (CART) analysis was used to identify non-HLA antibodies capable of differentiating rejection from non-rejection. FIG. 4 shows the CART analysis of sera obtained from adult cardiac (n=67 sera, FIG. 4A) and pediatric renal allograft transplant patients (n=129 sera, FIG. 4B). CART analysis of sera isolated from adult cardiac allograft transplant patients (n=67 sera) with a 1,000 MFI cut point was used in the analysis. The root node, LPHN1, that includes all 67 sera, 49% of which are rejection samples splits at an MFI<1000 into child nodes. As the algorithm progresses to terminal nodes, 65% of rejection samples are correctly identified (far right, dark boxes). Scale bar indicates association with rejection with lighter terminal node boxes correlating to non-rejection. Eight non-HLA antibodies (SNPRN, KRT18, LGALS3, KRT8, SNRPB2, DEXI, PLA2R1, Collagen II and GSTT1) were informative predictors of renal allograft rejection. The root node, SNPRN, that includes all 129 sera, 26% of which are rejection samples splits at an MFI<1000 into child nodes. As the algorithm progresses to terminal nodes, 56% of non-rejection samples are correctly identified (far left, darker boxes). Scale bar indicates association with rejection with lighter terminal node boxes correlating to non-rejection. Importantly, PLA2R1 and GSTT1 were also found to independently cluster among rejection samples, and three of the 8 (SNPRN, LGALS3, and DEXI) are newly identified herein.

Described hereinabove are non-HLA antibodies that are significantly associated with renal and cardiac allograft rejection. Using a protein microarray dotted with 9000 full length proteins, followed by gene ontological analysis and development of a high throughput multiplex bead array, 10 non-HLA antibodies were associated with cardiac allograft rejection (Table 2) and 18 non-HLA antibodies were identified as significantly associated with renal (pediatric and adult) transplant rejection (FIG. 1, Table 3). Two of these non-HLA antigens are newly described herein as associated with transplant rejection (LPHN1 and TG). Nine of these renal targets, DEXI, SHC3, SNPRN, LGALS3, CSF2, LGALS8 and STATE are newly described herein as associated with transplant rejection (FIG. 1, Table 3). The non-HLA antibodies are shown in Table 3 and FIG. 5. Some markers are found in multiple organs and are predictive in the CART analysis. Other markers are non-HLA Abs that sort independently. Still other markers include those non-H LA antibodies that sort together in the correlation matrix and are independent of other markers. Correlation matrix analysis identified a panel of non-HLA antibodies that can be used independently to predict rejection.

TABLE 4

| # | Antigen | Gene Name | Function | Reference |
|---|---|---|---|---|
| 1 | DEXI | dexa- | This gene encodes a member of the Ras superfamily of small GTPases and is induced by | |
| | | methasone- | dexamethasone. The encoded protein is an activator of G-protein signaling and acts as a | |
| | | induced | direct nucleotide exchange factor for Gi-Go proteins. This protein interacts with the neuronal | |
| | | transcript | nitric oxide adaptor protein CAPON, and a nuclear adaptor protein FE65, which interacts with | |
| | | | the Alzheimer's disease amyloid precursor protein. This gene may play a role in | |
| | | | dexamethasone-induced alterations in cell morphology, growth and cell-extracellular matrix | |
| | | | interactions. Epigenetic inactivation of this gene is closely correlated with resistance to | |
| | | | dexamethasone in multiple myeloma cells. Alternatively spliced transcript variants encoding | |
| | | | different isoforms have been found for this gene. Small GTPase. Negatively regulates the | |
| | | | transcription regulation activity of the APBB1/FE65-APP complex. | |
| 2 | EMCN | Endomucin | EMCN is a mucin-like sialoglycoprotein that interferes with the assembly of focal | |
| | | | adhesion complexes and inhibits interaction between cells and the extracellular | |
| | | | matrix (Kinoshita et al., 2001) | |
| 3 | SNRPN | Small | The protein encoded by this gene is one polypeptide of a small nuclear ribonucleoprotein | |
| | | Nuclear | complex and belongs to the snRNP SMB/SMN family. The protein plays a role in pre-mRNA | |
| | | Ribonucleo- | processing, possibly tissue-specific alternative spiicing events. Although individual snRNPs | |
| | | protein | are believed to recognize specific nucleic acid sequences through RNA-RNA base pairing, | |
| | | Polypeptide | the specific role of this family member is unknown. The protein arises from a bicistronic | |
| | | N (smith | transcript that also encodes a protein identified as the SNRPN upstream reading frame | |
| | | antigen | (SNURF). Multiple transcription initiation sites have been identified and extensive alternative | |
| | | core | splicing occurs in the 5′ untranslated region. Additional splice variants have been described | |
| | | sequence) | but sequences for the complete transcripts have not been determined. The 5′ UTR of this | |
| | | | gene has been identified as an imprinting center. Alternative splicing or deletion caused by a | |
| | | | translocation event in this paternally-expressed region is responsible for Angelman syndrome | |
| | | | or Prader-Willi syndrome due to parental imprint switch failure. | |
| 4 | NTRK1 | Neurotrophic | This gene encodes a member of the neurotrophic tyrosine kinase receptor (NTKR) family. This | |
| | | Tyrosine | kinase is a membrane-bound receptor that, upon neurotrophin binding, phosphorylates itself | |
| | | Kinase, | and members of the MAPK pathway. The presence of this kinase leads to cell differentiation | |
| | | Receptor, | and may play a role in specifying sensory neuron subtypes. Mutations in this gene have been | |
| | | Type 1 | associated with congenital insensitivity to pain, anhidrosis, self-mutilating behavior, mental | |
| | | | retardation and cancer. Alternate transcriptional splice variants of this gene have been found, | |
| | | | but only three have been characterized to date. Receptor tyrosine kinase involved in the | |
| | | | development and the maturation of the central and peripheral nervous systems through | |
| | | | regulation of proliferation, differentiation and survival of sympathetic and nervous neurons. | |
| 5 | ROR1 | Receptor | a receptor tyrosine kinase-like orphan receptor that modulates neurite growth in the centrai | |
| | | Tyrosine | nervous system. The encoded protein is a glycosyiated type I membrane protein that belongs | |
| | | Kinase-Like | to the ROR subfamily of cell surface receptors. It is a pseudokinase that lacks catalytic activity | |
| | | Orphan | and may interact with the non-canonical Wnt signalling pathway. This gene is highly | |
| | | Receptor 1 | expressed during early embryonic development but expressed at very low levels in adult | |
| | | | tissues. Increased expression of this gene is associated with B-cell chronic lymphocytic | |
| | | | leukaemia. Alternative splicing results in multiple transcript variants encoding different | |
| | | | isoforms. Tyrosine-protein kinase receptor whose role is not yet clear. A receptor tyrosine | |
| | | | kinase whose mutation in mouse cause skeletal disorders. It could be a biomarker for B-cell | |
| | | | lymphocytic leukemia and renal cancer. | |
| 6 | LGALS3 | lectin, | Galactose-specific lectin which binds igE, chemoattractant activity This protein plays a role in | |
| | | galactoside- | numerous cellular functions including apoptosis, innate immunity, cell adhesion and T-cell | |
| | | binding, | regulation. The protein exhibits antimicrobial activity against bacteria and fungi. a member of | |
| | | soluble, 3 | the galectin family of carbohydrate binding proteins. Members of this protein family have an | |
| | | | affinity for beta-galactosides. The encoded protein is characterized by an N-terminal proline- | |
| | | | rich tandem repeat domain and a single C-terminal carbohydrate recognition domain. This | |
| | | | protein can self-associate through the N-terminal domain allowing it to bind to multivalent | |
| | | | saccharide ligands. This protein localizes to the extracellular matrix, the cytoplasm and the | |
| | | | nucleus. This protein plays a role in numerous cellular functions including apoptosis, innate | |
| | | | immunity, cell adhesion and T-cell regulation. The protein exhibits antimicrobial activity | |
| | | | against bacteria and fungi. Alternate splicing results in multiple transcript variants. | |
| 7 | ANXA2R | annexin A2 | May act as a receptor for annexin II on marrow stromal cells to induce osteoclast formation. | |
| | | receptor | “Annexin A2 (A2) is a profibrinolytic receptor that binds both plasminogen and its activator, | |
| | | | tissue plasminogen activator (tPA), functioning as a cofactor for plasmin generation, and | |
| | | | localizing fibrinolytic activity to the EC surface. A2 is found on the surface membrane of ECs | |
| | | | and monocytes, and also on the brush-border membrane of placental syncytiotrophoblasts, all | |
| | | | of which are recognized targets of pathogenic antiphospholipid antibodies. Several lines of | |
| | | | evidence indicate that A2 acts as a tPA-dependent cofactor for cell surface plasmin | |
| | | | generation in vivo.” (Romay-Penabad 2009 Blood) | |
| 8 | IYD | iodotyrosine | This gene encodes an enzyme that catalyzes the oxidative NADPH-dependent | |
| | | deiodinase | deiodination of mono- and diiodotyrosine, which are the halogenated byproducts of thyroid | |
| | | | hormone production. The N-terminus of the protein functions as a membrane anchor. | |
| | | | Mutations in this gene cause congenital hypothyroidism due to dyshormonogenesis type 4, | |
| | | | which is also referred to as deiodinase deficiency, or iodotyrosine dehalogenase | |
| | | | deficiency, or thyroid hormonogenesis type 4. Alternative splicing results in multiple | |
| | | | transcript variants. | |
| 9 | EMCN | Endomucin | EMCN is a mucin-like sialoglycoprotein that interferes with the assembly of focal | |
| | | | adhesion complexes and inhibits interaction between cells and the extracellular | |
| | | | matrix (Kinoshita at al., 2001) | |
| 10 | FGER1 | fibroblast | Tyrosine-protein kinase that acts as cell-surface receptor for fibroblast growth factors and | |
| | | growth | plays an essential role in the regulation of embryonic development, cell proliferation, | |
| | | factor | differentiation and migration. The protein encoded by this gene is a member of the | |
| | | receptor 1 | fibroblast growth factor receptor (FGFR) family, where amino acid sequence is highly | |
| | | | conserved between members and throughout evolution. FGFR family members differ | |
| | | | from one another in their ligand affinities and tissue distribution. A full-length | |
| | | | representative protein consists of an extracellular region, composed of three | |
| | | | immunoglobulin-like domains, a single hydrophobic membrane-spanning segment and a | |
| | | | cytoplasmic tyrosine kinase domain. The extracellular portion of the protein interacts with | |
| | | | fibroblast growth factors, setting in motion a cascade of downstream signals, ultimately | |
| | | | influencing mitogenesis and differentiation. This particular family member binds both | |
| | | | acidic and basic fibroblast growth factors and is involved in limb induction. Mutations in | |
| | | | this gene have been associated with Pfeiffer syndrome, Jackson-Weiss syndrome, | |
| | | | Antley-Bixier syndrome, osteoglophonic dysplasia, and autosomal dominant Kailmann | |
| | | | syndrome 2. Chromosomal aberrations involving this gene are associated with stem cell | |
| | | | myeloproliferative disorder and stem cell leukemia lymphoma syndrome. Alternatively | |
| | | | spliced variants which encode different protein isoforms have been described: however, | |
| | | | not all variants have been fully characterized. | |
| 11 | HARS | Jo-1 | Aminoacyl-tRNA synthetases are a class of enzymes that charge tRNAs with their | |
| | | | cognative amino acids. The enzyme is responsible for the synthesis of histidyl-transfer | |
| | | | RNA, which is essential for the incorporation of histidine into proteins. It is a frequent | |
| | | | target of autoantibodies in the human autoimmune disease | |
| | | | polymyositis/dermatomyositis. | |
| 12 | SPN | Sialophorin, | The protein encoded by this gene is a major sialoglycoprotein found on the surface of | |
| | | CD43 | thymocytes, T lymphocytes, monocytes, granulocytes, and some B lymphocytes. It may | |
| | | | be part of a physiologic ligand-receptor complex involved in T-cell activation. During T-ceil | |
| | | | activation, this protein is actively removed from the T-cell-APC (antigen-presenting cell) | |
| | | | contact site, suggesting a negative regulatory role in adaptive immune response. Plays a | |
| | | | role in the physicochemical properties of the T-cell surface and in lectin binding. | |
| 13 | SSB | Sjogren | The protein encoded by this gene is involved in diverse aspects of RNA metabolism, | |
| | | syndrome | including binding and protecting poly(U) termini of nascent RNA polymerase III transcripts | |
| | | antigen B | from exonuclease digestion, processing 5′ and 3′ ends of pre-tRNA precursors, acting as | |
| | | (autoantigen | an RNA chaperone, and binding viral RNAs associated with hepatitis C virus. | |
| | | La) | Autoantibodies reacting with this protein are found in the sera of patients with Sjogren | |
| | | | syndrome and systemic lupus erythematosus. Alternative promoter usage results in two | |
| | | | different transcript variants which encode the same protein. | |
| 14 | IL-8 | interleukin | Member of the CXC chemokine family. This chemokine is one of the major mediators | |
| | | 8, | of the inflammatory response. This chemokine is secreted by several cell types. It | |
| | | CXCL8 | functions as a chemoattractant, and is also a potent angiogenic factor. It is a | |
| | | | chemotactic factor that attracts neutrophils, basophils, and T-cells, but not | |
| | | | rnonocytes. It is also involved in neutrophil activation. | |
| 15 | CCP | cyclic | From cross-sectional studies, anti-cyclic citrullinated peptide antibodies (anti-CCP) are | |
| | | citrullinated | present in approximately 1% of the population.1 ,2 Their presence has been associated | |
| | | peptide | with a high risk of subsequent development of rheumatoid arthritis (RA).3 ,4 Anti-CCP | |
| | | | antibodies, however, can be detected more than 10 years prior to disease onset.5 The | |
| | | | risk of progression to RA in anti-CCP-positive individuals from the general population has | |
| | | | been estimated at 5% over a 5-year period, 1 meaning that this test is unlikely to be of | |
| | | | value as a screening tool. In the years just prior to diagnosis, however, retrospective | |
| | | | studies have found the predictive value of CCP testing to be much higher, with a positive | |
| | | | predictive value (ppv) of 85% noted within 1.5 years of symptom onset. | |
| 16 | VEGFA | vascular | Growth factor active in angiogenesis, vasculogenesis and endothelial cell growth. | |
| | | endothelial | Induces endothelial cell proliferation, promotes cell migration, inhibits apoptosis and | |
| | | growth | induces permeabilization of blood vessels. Binds to the FLT1/VEGFR1 and | |
| | | factor A | KDR/VEGFR2 receptors, heparan sulfate and heparin. NRP1/Neuropilin-1 binds | |
| | | | isoforms VEGF-165 and VEGF-145. Isoform | |
| 17 | DQA1*01:02 | HLA-DQA1 | HLA-DQA1 belongs to the HLA class II alpha chain paralogues. The class II molecule is a | |
| | | | heterodimer consisting of an alpha (DQA) and a beta chain (DQB), both anchored in the | |
| | | | membrane. It plays a central role in the immune system by presenting peptides derived | |
| | | | from extracellular proteins. Class II molecules are expressed in antigen presenting cells | |
| | | | (APC: B Lymphocytes, dendritic cells, macrophages). Within the DQ molecule both the | |
| | | | alpha chain and the beta chain contain the polymorphisms specifying the peptide binding | |
| | | | specificities, resulting in up to four different molecules. | |
| 18 | LPHN1 | latrophilin 1 | Member of the latrophilin subfamily of G protein-coupled receptors (GPCR). | |
| | | | Latrophilins may function in both cell adhesion and signal transduction. | |
| 19 | TG | Thyro- | It is a glycoprotein homodimer produced predominantly by the thryroid gland. It acts | |
| | | globulin | as a substrate for the synthesis of thyroxine and triiodothyronine as well as the | |
| | | | storage of the inactive forms of thyroid hormone and iodine | |
| 20 | FGF2 | fibroblast | FGF family members bind heparin and possess broad mitogenic and angiogenic | |
| | | growth | activities. This protein has been implicated in diverse biological processes, such as limb | |
| | | factor | and nervous system development, wound healing, and tumor growth. The mRNA for this | |
| | | receptor 2 | gene contains multiple polyadenylation sites, and is alternatively translated from non-AUG | |
| | | | (CUG) and AUG initiation codons, resulting in five different isoforrns with distinct | |
| | | | properties. | |
| 21 | CSF2 | Colony | Cytokine that controls the production, differentiation, and function of granulocytes | |
| | | stimulating | and macrophages. | |
| | | factor 2 | | |
| 22 | NGF | Nerve | This gene is a member of the NGF-beta family and encodes a secreted protein | |
| | | growth | which homodimerizes and is incorporated into a larger complex. This protein has | |
| | | factor | growth stimulating activity and the complex is involved in the regulation of growth. | |
| 23 | LGALS8 | lectin, | Galectins are beta-galactoside-binding animal lectins with conserved carbohydrate | |
| | | galactoside- | recognition domains. The galectins have been implicated in development, | |
| | | binding, | differentiation, cell-cell adhesion, cell-matrix interaction, growth regulation, apoptosis, | |
| | | soluble, 8 | and RNA splicing. This gene is widely expressed in tumoral tissues and seems to be | |
| | | | involved in integrin-like cell interactions. Alternatively spliced transcript variants | |
| | | | encoding different isoforms have been identified. | |
| 24 | LGALS1 | lectin, | The galectins are a family of beta-galactoside-binding proteins implicated in | |
| | | galactoside- | modulating cell-cell and cell-matrix interactions. This gene product may act as an | |
| | | binding, | autocrine negative growth factor that regulates cell proliferation. | |
| | | soluble, 1 | | |
| 25 | MAPK1 | Mitogen- | MAP kinases, also known as extracellular signal-regulated kinases (ERKs), act as an | |
| | | Activated | integration point for multiple biochemical signals, and are involved in a wide variety | |
| | | Protein | of cellular processes such as proliferation, differentiation, transcription regulation and | |
| | | Kinase 1 | development. The activation of this kinase requires its phosphorylation by upstream | |
| | | | kinases. | |
| 26 | SDF1B | C-X-C | CXCL12, ligand for the G-protein coupled receptor, chemokine (C-X-C motif) | |
| | | Motif | receptor 4, and plays a role in many diverse cellular functions, including | |
| | | Chemokine | embryogenesis, immune surveillance, inflammation response, tissue homeostasis, | |
| | | Ligand 12 | and tumor growth and metastasis. | |
| 27 | VWF | Von | A glycoprotein involved in hemostasis. The encoded pre-protein is proteolytically | |
| | | Willebrand | processed following assembly into large multimeric complexes. | |
| | | Factor | | |
| 28 | STAT6 | Signal | A member of the STAT family of transcription factors. In response to cytokines and | |
| | | Transducer | growth factors, STAT family members are phosphorylated by the receptor associated | |
| | | And Acti- | kinases. | |
| | | vator Of | | |
| | | Transcrip- | | |
| | | tion 6 | | |
| 29 | SHC3 | SHC | Among its related pathways are immune system and signaling by GPCR. GO | |
| | | Adaptor | annotations related to this gene include protein kinase binding and receptor tyrosine | |
| | | Protein 3 | kinase binding. | |
| 30 | GNG5 | G Protein | G proteins are trimeric (alpha-beta-gamma) membrane-associated proteins that | |
| | | Subunit | regulate flow of information from cell surface receptors to a variety of internal | |
| | | Gamma 5 | metabolic effectors. Interaction of a G protein with its activated receptor promotes | |
| | | | exchange of GTP for GDP that is bound to the alpha subunit. | |
| 31 | IL18R1 | Interleukin | The protein encoded by this gene is a cytokine receptor that belongs to the | |
| | | 18 | interleukin 1 receptor family. | |
| | | Receptor 1 | | |
| 32 | IL-21 | interleukin | The encoded protein plays a role in both the innate and adaptive immune | Sigdel, |
| | | 21 | responses by inducing the differentiation, proliferation and activity of multiple target | JASN, |
| | | | cells including macrophages, natural killer cells, B cells and cytotoxic T cells. | 2012 |
| | | | Cytokine with immunoregulatory activity. May promote the transition between innate | |
| | | | and adaptive immunity. Induces the production of IgG(1) and IgG(3) in B-cells (By | |
| | | | similarity). May play a role in proliferation and maturation of natural killer (NK) cells | |
| | | | in synergy with IL15. May regulate proliferation of mature B- and T-cells in response | |
| | | | to activating stimuli. In synergy with IL15 and IL18 stimulates interferon gamma | |
| | | | production in T-cells and NK cells. During T-cell mediated immune response may | |
| | | | inhibit dendritic cells (DC) activation and maturation. | |
| 33 | VIM | Vimentin | Member of the intermediate filament family. Intermediate filamentents, along with | Jurcevic, |
| | | | microtubules and actin microfilaments, make up the cytoskeleton. It is responsible for | Transplanta- |
| | | | maintaining cell shape, integrity of the cytoplasm, and stabilizing cytoskeletal | tion, 2001; |
| | | | interactions. It is also involved in the immune response, and controls the transport of | Besarani, |
| | | | low-density lipoprotein (LDL)-derived cholesterol from a lysosome to the site of | Transplanta- |
| | | | esterification. It functions as an organizer of a number of critical proteins involved in | tion, 2014 |
| | | | attachment, migration, and cell signaling. Among its related pathways are ERK | |
| | | | Signaling. | |
| 34 | TUBA1B | tubulin, | Tubulin is the major constituent of microtubules. It binds two moles of GTP, one at an | Hoffman, |
| | | alpha 1b | exchangeable site on the beta chain and one at a non-exchangeable | Auto- |
| | | | site on the alpha chain. *Microtubules are cylindrical tubes of 20-25 nm in | immunity, |
| | | | diameter. They are composed of protofilaments which are in turn composed of | 2016; |
| | | | alpha- and beta-tubulin polymers. Each microtubule is polarized, at one end alpha- | Bilalic, |
| | | | subunits are exposed (−) and at the other beta-subunits are exposed (+). | J Proteom |
| | | | Microtubules act as a scaffold to determine cell shape, and provide a backbone for | Res, 2010 |
| | | | cell organelles and vesicles to move on, a process that requires motor proteins. The | |
| | | | major microtubule motor proteins are kinesin, which generally moves towards the (+) | |
| | | | end of the microtubule, and dynein, which generally moves towards the (−) end. | |
| | | | Microtubules also form the spindle fibers for separating chromosomes during mitosis. | |
| 35 | Tubulin | Tubulin | Tubulin is the major constituent of microtubules. It binds two moles of GTP, one at an | Hoffman, |
| | | | exchangeable site on the beta chain and one at a non-exchangeable | Auto- |
| | | | site on the alpha chain. *Microtubules are cylindrical tubes of 20-25 nm in diameter. They | immunity, |
| | | | are composed of protofilaments which are in turn composed of alpha- and beta-tubulin | 2016; |
| | | | polymers. Each microtubule is polarized, at one end alpha-subunits are exposed (−) and at the | Bilalic, |
| | | | other beta-subunits are exposed (+). Microtubules act as a scaffold to determine cell shape, | J Proteom |
| | | | and provide a backbone for cell organelles and vesicles to move on, a process that requires | Res, 2010 |
| | | | motor proteins. The major microtubule motor proteins are kinesin, which generally moves | |
| | | | towards the (+) end of the microtubule, and dynein, which generally moves towards the (−) | |
| | | | end. Microtubules also form the spindle fibers for separating chromosomes during mitosis. | |
| 36 | APOA1 | apolipo- | This gene encodes apolipoprotein A-I, which is the major protein component of high density | Ziegler, |
| | | protein | lipoprotein (HDL) in plasma. The protein promotes cholesterol efflux from tissues to the liver | Transplanta- |
| | | A-I | for excretion, and it is a cofactor for lecithin cholesterolacyltransferase (LCAT) which is | tion, 2011 |
| | | | responsible for the formation of most plasma cholesteryl esters. This gene is closely linked | |
| | | | with two other apolipoprotein genes on chromosome 11. Defects in this gene are associated | |
| | | | with HDL deficiencies, including Tangier disease, and with systemic non-neuropathic | |
| | | | amyloidosis. | |
| 37 | ICAM1 | Intracellular | This gene encodes a cell surface glycoprotein which is typically expressed on endothelial cells | Lawson, |
| | | Adhesion | and cells of the immune system. It binds to integrins of type CD11a / CD18, or CD11b / CD18 | Transplanta- |
| | | Molecule 1 | and is also exploited by Rhinovirus as a receptor. | tion, 2005 |
| 38 | Myosin | Myosin | Muscle myosin is a hexameric protein containing 2 heavy chain subunits, 2 alkali light chain | Kalache, |
| | | | subunits, and 2 regulatory light chain subunits. This gene encodes the beta (or slow) heavy | Journal of |
| | | | chain subunit of cardiac myosin. It is expressed predominantly in normal human ventricle. It is | Immu- |
| | | | also expressed in skeletal muscle tissues rich in slow-twitch type I muscle fibers. Changes in | nology, |
| | | | the relative abundance of this protein and the alpha (or fast) heavy subunit of cardiac myosin | 2011; Nath, |
| | | | correlate with the contractile velocity of cardiac muscle. Its expression is also altered during | Hum |
| | | | thyroid hormone depletion and hemodynamic overloading. Mutations in this gene are associated | Immunol, |
| | | | with familial hypertrophic cardiomyopathy, myosin storage myopathy, dilated cardiomyopathy, | 2010 |
| | | | and Laing early-onset distal myopathy. | |
| 39 | KRT8 | Cytokeratin | This gene is a member of the type II keratin family clustered on the long arm of chromosome | Alvarez- |
| | | 8 | 12. Type I and type II keratins heteropolymerize to form intermediate-sized filaments in the | Marquez, |
| | | | cytoplasm of epithelial cells. The product of this gene typically dimerizes with keratin 18 to | Hum |
| | | | form an intermediate filament in simple single-layered epithelial cells. This protein plays a | Immunol, |
| | | | role in maintaining cellular structural integrity and also functions in signal transduction and | 2008 |
| | | | cellular differentiation. Mutations in this gene cause cryptogenic cirrhosis. Altematively | |
| | | | spliced transcript variants have been found for this gene. | |
| 40 | KRT18 | Cytokeratin | | Alvarez- |
| | | 18 | | Marquez, |
| | | | | Hum |
| | | | | Immunol, |
| | | | | 2008 |
| 41 | HSPB1 | Heat shock | The protein encoded by this gene is induced by environmental stress and developmental | Pockley, |
| | | protein | changes. The encode protein is involved in stress resistance and actin organization and translo- | Transplanta- |
| | | beta-1 | cates from the cytoplasm to the nucleus upon stress induction. Defects in this gene are a cause | tion, 2001 |
| | | | of Charcot-Marie-Tooth disease type 2F (CMT2F) and distal hereditary motor neuropathy | |
| 42 | FLRT2 | Leucine-rich | This gene encodes a member of the fibronectin leucine rich transmembrane protein (FLRT) | Shirai, |
| | | repeat trans- | family. FLRT family members may function in cell adhesion and/or receptor signalling. Their | Arthritis Res |
| | | membrane | protein structures resemble small leucine-rich proteoglycans found in the extracellular matrix. | Ther, 2012 |
| | | protein | | |
| | | FLRT2 | | |
| 43 | Collagen VI | Collagen VI | Extracellular matrix proteins and have a triple-helical domain as their common structural | Nath, |
| | | | element. Collagen VI is a major structural component of microfibrils. The basic structural unit | Transplanta- |
| | | | of collagen VI is a heterotrimer of the alpha1(VI), alpha2(VI), and alpha3(VI) chains. | tion, 2011 |
| 44 | Collagen I | Collagen I | Type I is a fibril-forming collagen found in most connective tissues and is abundant in bone, | Nath, |
| | | | cornea, dermis and tendon. Mutations in this gene are associated with osteogenesis imperfecta | Transplanta- |
| | | | types I-IV, Ehlers-Danlos syndrome type VIIA, Ehlers-Danlos syndrome Classical type, Caffey | tion, 2011 |
| | | | Disease and idiopathic osteoporosis. | |
| 45 | Collagen II | Collagen II | A fibrillar collagen found in cartilage and the vitreous humor of the eye. Mutations in this gene | Nath, |
| | | | are associated with achondrogenesis, chondrodysplasia, early onset familial osteoarthritis, SED | Transplanta- |
| | | | congenita, Langer-Saldino achondrogenesis, Kniest dysplasia, Stickler syndrome type I, and | tion, 2011 |
| | | | spondyloepimetaphyseal dysplasia Strudwick type. | |
| 46 | Collagen III | Collagen III | a fibrillar collagen that is found in extensible connective tissues such as skin, lung, uterus, | Nath, |
| | | | intestine and the vascular system, frequently in association with type I collagen. Mutations in | Transplanta- |
| | | | this gene are associated with Ehlers-Danlos syndrome types IV, and with aortic and arterial | tion, 2011 |
| | | | aneurysms. | |
| 47 | Collagen IV | Collagen IV | Type IV collagen, the major structural component of basement membranes, is a multimeric | Nath, |
| | | | protein composed of 3 alpha subunits. These subunits are encoded by 6 different genes, alpha | Transplanta- |
| | | | 1 through alpha 6, each of which can form a triple helix structure with 2 other subunits to form | tion, 2011 |
| | | | type IV collagen. This gene encodes alpha 3. In the Goodpasture syndrome, autoantibodies | |
| | | | bind to the collagen molecules in the basement membranes of alveoli and glomeruli. | |
| 48 | Collagen V | Collagen V | This gene encodes an alpha chain for one of the low abundance fibrillar collagens. Fibrillar | Nath, |
| | | | collagen molecules are trimers that can be composed of one or more types of alpha chains. | Transplanta- |
| | | | TypeV collagen is found in tissues containing type I collagen and appears to regulate the | tion, 2011; |
| | | | assembly of heterotypic fibers composed of both type I and type V collagen. | Burlingham, |
| | | | | J Clin Invest, |
| | | | | 2007 |
| 49 | GSTT1 | Glutathione | The protein encoded by this gene, glutathione S-transferase (GST) theta 1 (GSTT1), is a | Aguilera, |
| | | S- | member of a superfamily of proteins that catalyze the conjugation of reduced glutathione to a | Clin Exp |
| | | Transferase | variety of electrophilic and hydrophobic compounds. Human GSTs can be divided into five | Immunol, |
| | | theta-1 | main classes: alpha, mu, pi, theta, and zeta. The theta class includes GSTT1 and GSTT2, | 2001; |
| | | | GSTT1 and GSTT2 share 55% amino acid sequence identity and both may play an important | Aguilera, |
| | | | role in human carcinogenesis. The GSTT1 and GSTT2 genes have a similar structure, being | Transplant |
| | | | composed of five exons with identical exon/intron boundaries. This GSTT1 gene is hapiotype- | Proc, 2005 |
| | | | specific and is absent from 38% of the population. Alternative splicing of this gene results in | |
| | | | multiple transcript variants. Two related pseudogenes, which are also located on chromosome | |
| | | | 22, have been identified. | |
| 50 | ENO1 | Alpha- | Multifunctional enzyme that, as well as its role in glycolysis, plays a part in various processes | Murtas, Clin |
| | | enolase | such as growth control, hypoxia tolerance and allergic responses. Stimulates immunoglobulin | J Am Soc |
| | | | production. | Nephrol, |
| | | | | 2012 |
| 51 | Actin | Actin | | Alvarez- |
| | | | | Marquez, |
| | | | | Hum |
| | | | | Immunol, |
| | | | | 2008 |
| 52 | GAPDH | Glyceralde- | Has both glyceraldehyde-3-phosphate dehydrogenase and nitrosylase activities, thereby playing | Buse, |
| | | hyde-3- | a role in glycolysis and nuclear functions, respectively. Participates in nuclear events including | Electro- |
| | | phosphate | transcription, RNA transport, DNA replication and apoptosis. Nuclear functions are probably | pheresis, |
| | | dehydro- | due to the nitrosylase activity that mediates cysteine S-nitrosylation of nuclear target proteins | 2008 |
| | | genase | such as SIRT1, HDAC2 and PRKDC. Modulates the organization and assembly of the cyto- | |
| | | | skeleton. Facilitates the CHP1-dependent microtubule and membrane associations through its | |
| | | | ability to stimulate the binding of CHP1 to microtubules (By similarity). | |
| 53 | LMNA | Prelamin- | Lamins are components of the nuclear lamina, a fibrous layer on the nucleoplasrnic side of the | Cortes, |
| | | A/C | inner nuclear membrane, which is thought to provide a framework for the nuclear envelope and | Transplant |
| | | | may also interact with chromatin. | Proc., 2009; |
| | | | | Bilalic, J |
| | | | | Proteome |
| | | | | Res, 2010 |
| 54 | VCL | Vinculin | Actin filament (F-actin)-binding protein involved in cell-matrix adhesion and cell-cell adhesion. | Wilke, Herz, |
| 55 | NCL | nucleolin | the major nucleolar protein of growing eukaryotic cells. It is found associated with intra- | 1995 |
| | | | nucleolar chromatin and pre-ribosomal particles. It induces chromatin decondensation by | Qin, |
| | | | binding to histone H1. | Transplanta- |
| | | | | tion, 2011 |
| 56 | PRKCH | Protein | calcium-independent protein kinase C activity, involved in the regulation of cell differentiation | Sutherland, |
| | | kinase | in keratinocytes and pre-B cell receptor, mediates regulation of epithelial tight junction integrity | Kidney |
| | | C, eta | | |
| 57 | PRKCZ | protein | A member of the PKC family of serine/threonine kinases which are involved in a variety of | international, |
| | | kinase | cellular processes such as proliferation, differentiation and secretion. Unlike the classical PKC | 2009 |
| | | C, zeta | isoenzymes which are calcium-dependent. PKC zeta exhibits a kinase activity which is indepen- | Sutherland, |
| | | | dent of calcium and diacylglycerol but not of phosphatidylserine. Furthermore, it is insensitive | Kidney |
| | | | to typical PKC inhibitors and cannot be activated by phorbol ester. Unlike the classical PKC | international, |
| | | | isoenzymes, it has only a single zinc finger module. These structural and biochemical | 2009 |
| | | | properties indicate that the zeta subspecies is related to, but distinct from other isoenzymes of | |
| | | | PKC. Alternative splicing results in multiple transcript variants encoding different isoforms. | |
| 58 | PLA2R1 | phospho- | This gene represents a phospholipase A2 receptor. The encoded protein likely exists as both a | Kanigicherla, |
| | | lipase A2 | transmembrane form and a soluble form. The transmembrane receptor may play a role in | Kidney |
| | | receptor 1, | clearance of phospholipase A2, thereby inhibiting its action. Polymorphisms at this locus have | Int, 2013 |
| | | 180kDa | been associated with susceptibility to idiopathic membranous nephropathy. Alternatively | |
| | | | spliced transcript variants encoding different isoforms have been identified. | |
| 59 | FN1 | fibronectin | This gene encodes fibronectin, a glycoprotein present in a soluble dimeric form in plasma, and | Angaswarny, |
| | | 1 | in a dimeric or multimeric form at the cell surface and in extracellular matrix. Fibronectin is | Am J |
| | | | involved in cell adhesion and migration processes including embryogenesis, wound healing, | Transplant, |
| | | | blood coagulation, host defense, and metastasis. The gene has three regions subject to | 2014 |
| | | | alternative splicing, with the potential to produce 20 different transcript variants. However, the | |
| | | | full-length nature of some variants has not been determined. | |
| 60 | PECR | Peroxisomal | PECR (Peroxisomal Trans-2-Enoyl-CoA Reductase) is a Protein Coding gene. Among its | Dinavahi, |
| | | trans-2- | related pathways are Peroxisome and Mitochondrial LC-Fatty Acid Beta-Oxidation. GO | J Am |
| | | enoyl-CoA | annotations related to this gene include receptor binding and trans-2-enoyl-CoA reductase | Soc Nephrol, |
| | | Reductase | (NADPH) activity. An important paralog of this gene is DECR2. | 2011 |
| 61 | HSPG2, | heparan | This gene encodes the perlecan protein, which consists of a core protein to which three long | Joosten, |
| | Perlecan | sulfate | chains of glycosaminoglycans (heparan sulfate or chondroitin sulfate) are attached. The | Am J |
| | | proteo- | perlecan protein is a large multidomain proteoglycan that binds to and cross-links many extra- | Transplant, |
| | | glycan 2 | cellular matrix components and cell-surface molecules. It has been shown that this protein | 2005 |
| | | | interacts with laminin, prolargin, collagen type IV, FGFBP1, FBLN2, FGF7 and transthyretin, | |
| | | | etc., and it plays essential roles in multiple biological activities. Perlecan is a key component | |
| | | | of the vascular extra-cellular matrix, where it helps to maintain the endothelial barrier | |
| | | | function. It is a potent inhibitor of smooth muscle cell proliferation and is thus thought to help | |
| | | | maintain vascular homeostasis. It can also promote growth factor (e.g., FGF2) activity and | |
| | | | thus stimulate endothelial growth and re-generation. It is a major component of basement | |
| | | | membranes, where it is involved in the stabilization of other molecules as well as being | |
| | | | involved with glomerular permeability to macro molecules and cell adhesion. Mutations | |
| | | | in this gene cause Schwartz-Jampel syndrome type 1, Silverman-Handmaker type of | |
| | | | dyssegmental dysplasia, and tardive dyskinesia. Alternative splicing of this gene results | |
| | | | in multiple transcript variants. | |
| 62 | AGRN | Agrin | This gene encodes one of several proteins that are critical in the development of the | Joosten, |
| | | | neuromuscular junction (NMJ), as identified in mouse knock-out studies. The encoded protein | Am J |
| | | | contains several laminin G, Kazal type serine protease inhibitor, and epidermal growth factor | Transplant, |
| | | | domains. Additional post-translational modifications occur to add glycosaminoglycans and | 2005 |
| | | | disulfide bonds. In one family with congenital myasthenic syndrome affecting limb-girdle | |
| | | | muscles, a mutation in this gene was found. Alternative splicing results in multiple transcript | |
| | | | variants encoding different isoforms. | |
| 63 | ATP5B | ATP | This gene encodes a subunit of mitochondrial ATP synthase. Mitochondrial ATP synthase | Hsu, |
| | | synthase, H+ | catalyzes ATP synthesis, utilizing an electrochemical gradient of protons across the inner | Arthritis |
| | | transporting, | membrane during oxidative phosphorylation. ATP synthase is composed of two linked multi- | Rheum, |
| | | mito- | subunit complexes: the soluble catalytic core, F1, and the membrane-spanning component, Fo, | 2006 |
| | | chondrial | comprising the proton channel. The catalytic portion of mitochondrial ATP synthase consists | |
| | | F1 complex, | of 5 different subunits (alpha, beta, gamma, delta, and epsilon) assembled with a stoichiometry | |
| | | beta | of 3 alpha, 3 beta, and a single representative of the other 3. The proton channel consists of three | |
| | | polypeptide | main subunits (a, b, c). This gene encodes the beta subunit of the catalytic core. | |
| 64 | NPHS1 | nephrosis 1, | This gene encodes a member of the immunoglobulin family of cell adhesion molecules that | Kuusniemi, |
| | | congenital, | functions in the glornerular filtration barrier in the kidney. The gene is primarily expressed in | Transplanta- |
| | | Finnish type | renal tissues, and the protein is a type-1 transmembrane protein found at the slit diaphragm of | tion, 2007 |
| | | (nephrin) | glomerular podocytes. The slit diaphragm is thought to function as an ultrafilter to exclude | |
| | | | albumin and other plasma macromolecules in the formation of urine. Mutations in this gene | |
| | | | result in Finnish-type congenital nephrosis 1, characterized by severe proteinuria and loss of the | |
| | | | slit diaphragm and foot processes. | |
| 65 | APOL2 | apolipo- | This gene is a member of the apolipoprotein L gene family. The encoded protein is found in the | Delville, Sci |
| | | protein L, 2 | cytoplasm, where it may affect the movement of lipids or allow the binding of lipids to | Transl Med, |
| | | | organelles. Two transcript variants encoding the same protein have been found for this gene. | 2014 |
| 66 | CD40 | CD40 | This gene is a member of the TNF-receptor superfamily. The encoded protein is a receptor on | Delville, Sci |
| | | molecule, | antigen-presenting cells of the immune system and is essential for mediating a broad variety of | Transl Med, |
| | | TNF | immune and inflammatory responses including T cell-dependent immunoglobulin class switch- | 2014 |
| | | receptor | ing, memory B cell development, and germinal center formation. AT-hook transcription factor | |
| | | superfamily | AKNA is reported to coordinately regulate the expression of this receptor and its ligand, which | |
| | | member 5 | may be important for homotypic cell interactions. Adaptor protein TNFR2 interacts with this | |
| | | | receptor and serves as a mediator of the signal transduction. The interaction of this receptor | |
| | | | and its ligand is found to be necessary for amyloid-beta-induced microglial activation, and thus | |
| | | | is thought to be an early event in Alzheimer disease pathogenesis. Mutations affecting this | |
| | | | gene are the cause of autosomal recessive hyper-igM immunodeficiency type 3 (HIGM3). | |
| | | | Multiple alternatively spliced transcript variants of this gene encoding distinct isoforms have | |
| | | | been reported. | |
| 67 | CGB5 | chorionic | This gene is a member of the glycoprotein hormone beta chain family and encodes the beta 5 | Delville, Sci |
| | | gonadotro- | subunit of chorionic gonadotropin (CG). Glycoprotein hormones are heterodimers consisting of | Transl Med, |
| | | pin, beta | a common alpha subunit and an unique beta subunit which confers biological specificity. CG is | 2014 |
| | | polypeptide | produced by the trophoblastic cells of the placenta and stimulates the ovaries to synthesize the | |
| | | 5 | steroids that are essential for the maintenance of pregnancy. The beta subunit of CG is encoded | |
| | | | by 6 genes which are arranged in tandem and inverted pairs on chromosome 19q13.3 and | |
| | | | contiguous with the luteinizing hormone beta subunit gene. | |
| 68 | FAS | Fas cell | The protein encoded by this gene is a member of the TNF-receptor superfamily. This receptor | Delville, Sci |
| | | surface | contains a death domain. It has been shown to play a central role in the physiological regulation | Transl Med, |
| | | death | of programmed cell death, and has been implicated in the pathogenesis of various malignancies | 2014 |
| | | receptor | and diseases of the immune system. The interaction of this receptor with its ligand allows the | |
| | | | formation of a death-inducing signaling complex that includes Fas-associated death domain | |
| | | | protein (FADD), caspase 8, and caspase 10. The autoproteolytic processing of the caspases in | |
| | | | the complex triggers a downstream caspase cascade, and leads to apoptosis. This receptor has | |
| | | | been also shown to activate NF-kappaB, MAPK3/ERK1, and MAPK8/JNK, and is found to be | |
| | | | involved in transducing the proliferating signals in normal diploid fibroblast and T cells. Several | |
| | | | alternatively spliced transcript variants have been described, some of which are candidates | |
| | | | for nonsense-mediated mRNA decay (NMD). The isoforms lacking the transmembrane | |
| | | | domain may negatively regulate the apoptosis mediated by the full length isoform. | |
| 69 | P2RY11 | purinergic | The product of this gene belongs to the family of G-protein coupled receptors. This family has | Delville, Sci |
| | | receptor | several receptor subtypes with different pharmacological selectivity, which overlaps in some | Transl Med, |
| | | P2Y, | cases, for various adenosine and uridine nucleotides. This receptor is coupled to the stimulation | 2014 |
| | | G-protein | of the phosphoinositide and adenylyl cyclase pathways and behaves as a selective purinoceptor. | |
| | | coupled, 11 | Naturally occuring read-through transcripts, resulting from intergenic splicing between this gene | |
| | | | and an immediately upstream gene (PPAN, encoding peter pan homolog), have been found. | |
| | | | The PPAN-P2RY11 read-through mRNA is ubiquitously expressed and encodes a fusion | |
| | | | protein that shares identity with each individual gene product. | |
| 70 | PTPRO | Receptor- | This gene encodes a member of the R3 subtype family of receptor-type protein tyrosine phos- | Delville, Sci |
| | | type | phatases. These proteins are localized to the apical surface of polarized cells and may have | Transl Med, |
| | | Tyrosine- | tissue-specific functions through activation of Src family kinases. This gene contains two | 2014 |
| | | protein | distinct promoters, and alternatively spliced transcript variants encoding multiple isoforms have | |
| | | Phosphatase | been observed. The encoded proteins may have multiple isoform-specific and tissue-specific | |
| | | U | functions, including the regulation of osteoclast production and activity, inhibition of cell | |
| | | | proliferation and facilitation of apoptosis. This gene is a candidate tumor suppressor, and | |
| | | | decreased expression of this gene has been observed in several types of cancer. | |
| 71 | SNRPB2 | small nuclear | The protein encoded by this gene associates with stem loop IV of U2 small nuclear | Delville, Sci |
| | | ribonucleo- | ribonucleoprotein (U2 snRNP) in the presence of snRNP-A′. The encoded protein may play a | Transl Med, |
| | | protein | role in pre-mRNA splicing. Autoantibodies from patients with systemic lupus erythematosus | 2014 |
| | | polypeptide | frequently recognize epitopes on the encoded protein. Two transcript variants encoding the | |
| | | B | same protein have been found for this gene. | |
| 72 | CXCL11 | chemokine | Chemokines are a group of small (approximately 8 to 14 kD), mostly basic, structurally related | Sigdel, J |
| | | (C-X-C | molecules that regulate cell trafficking of various types of leukocytes through interactions with | Am Soc |
| | | motif) | a subset of 7-transmembrane, G protein-coupled receptors. Chemokines also play fundamental | Nephrol, |
| | | ligand 11 | roles in the development, homeostasis, and function of the immune system, and they have | 2012 |
| | | | effects on cells of the central nervous system as well as on endothelial cells involved in angio- | |
| | | | genesis or angiostasis. Chemokines are divided into 2 major subfamilies, CXC and CC. This | |
| | | | antimicrobial gene is a CXC member of the chemokine superfamily. Its encoded protein | |
| | | | induces a chemotactic response in activated T-cells and is the dominant ligand for CXC | |
| | | | receptor-3. The gene encoding this protein contains 4 exons and at least three polyadenylation | |
| | | | signals which might reflect cell-specific regulation of expression. IFN-gamma is a potent | |
| | | | inducer of transcription of this gene. Two transcript variants encoding different isoforms | |
| | | | have been found for this gene. | |
| 73 | GDNF | glial cell | This gene encodes a highly conserved neurotrophic factor. The recombinant form of this protein | Sigdel, J |
| | | derived | was shown to promote the survival and differentiation of dopaminergic neurons in culture, and | Am Soc |
| | | neurotrophic | was able to prevent apoptosis of motor neurons induced by axotomy. The encoded protein is | Nephrol, |
| | | factor | processed to a mature secreted form that exists as a homodimer. The mature form of the protein | 2012 |
| | | | is a ligand for the product of the RET (rearranged during transfection) protooncogene. Multiple | |
| | | | transcript variants encoding different isoforms have been found for this gene. Mutations in this | |
| | | | gene may be associated with Hirschsprung disease. | |
| 74 | IFNG | Interferon | This gene encodes a member of the type II interferon family. The protein encoded is a soluble | Sigdel, J |
| | | Gamma | cytokine with antiviral, immunoregulatory and anti-tumor properties and is a potent activator of | Am Soc |
| | | | macrophages. Mutations in this gene are associated with aplastic anemia. | Nephrol, |
| | | | | 2012 |
| 75 | CXCL9 | C-X-C | This antimicrobial gene encodes a protein thought to be involved in T cell trafficking. The | Sigdel, J |
| | | Motif | encoded protein binds to C-X-C motif chemokine 3 and is a chemoattractant for lymphocytes | Am Soc |
| | | Chemokine | but not for neutrophils. | Nephrol, |
| | | 9 | | 2012 |
| 76 | EDIL3 | EGF-like | The protein encoded by this gene is an integrin ligand. It plays an important role in mediating | Jackson, J |
| | | repeats and | angiogenesis and may be important in vessel wall remodeling and development. It also | Am Soc |
| | | discoidin | influences endothelial cell behavior. | Nephrol, |
| | | I-like | | 2015 |
| | | domains 3 | | |
| 77 | ENG | endoglin | This gene encodes a homodimeric transmembrane protein which is a major glycoprotein of the | Jackson, J |
| | | | vascular endothelium. This protein is a component of the transforming growth factor beta | Am Soc |
| | | | receptor complex and it binds to the beta1 and beta3 peptides with high affinity. Mutations in | Nephrol, |
| | | | this gene cause hereditary hemorrhagic telangiectasia, also known as Osler-Rendu-Weber | 2015 |
| | | | syndrome 1, an autosornal dominant multisystemic vascular dysplasia. This gene may also be | |
| | | | involved in preeclampsia and several types of cancer. Alternatively spliced transcript variants | |
| | | | encoding different isoforms have been found for this gene. | |
| 78 | ICAM4 | intercellular | This gene encodes the Landsteiner-Wiener (LW) blood group antigen(s) that belongs to the | Jackson, J |
| | | adhesion | immunoglobulin (Ig) superfamily, and that shares similarity with the intercellular adhesion | Am Soc |
| | | molecule 4 | molecule (ICAM) protein family. This ICAM protein contains 2 Ig-like C2-type domains and | Nephrol, |
| | | (Land- | binds to the leukocyte adhesion LFA-1 protein. The molecular basis of the LW(A)/LW(B) | 2015 |
| | | steiner- | blood group antigens is a single aa variation at position 100; Gln-100 = LW(A) and Arg- | |
| | | Wiener | 100 = LW(B). Alternative splicing results in multiple transcript variants encoding distinct | |
| | | blood group) | isoforms. | |
| 79 | FLT3LG | fms-related | Dendritic cells (DCs) provide the key link between innate and adaptive immunity by | Jackson, J |
| | | tyrosine | recognizing pathogens and priming pathogen-specific immune responses. FLT3LG controls | Am Soc |
| | | kinase 3 | the development of DCs and is particularly important for plasmacyloid DCs and CD8 (see | Nephrol, |
| | | ligand | MIM 186910)-positive classical DCs and their CD103 (ITGAE; MIM 604682)-positive tissue | 2015 |
| | | | counterparts | |

### Example 2. Validation of Non-HLA Antibodies Associated with Cardiac Allograft Rejection

Additional analysis of the non-HLA antigens described above was performed as described in C. L. Butler et al., Am J Transplant. 2020; 00:1-13. The study of adult cardiac allograft recipients (samples: n=477 no rejection; n=69 rejection) identified 18 non-HLA antibodies associated with rejection (P<0.1) including 4 newly identified non-HLA antigenic targets (DEXI, EMCN, LPHN1, and SSB). CART analysis showed 5/18 non-HLA antibodies distinguished rejection vs nonrejection. Antibodies to 4/18 non-HLA antigens synergize with HLA donor-specific antibodies and significantly increase the odds of rejection (P<0.1). The non-HLA panel was validated using an independent adult cardiac transplant cohort (n=21 no rejection; n=42 rejection, >1R) with an area under the curve of 0.87 (P<0.05) with 92.86% sensitivity and 66.67% specificity. The results confirm that multiplex bead array assessment of non-HLA antibodies identifies cardiac transplant recipients at risk of rejection.

Table 5 lists targets identified through the additional analysis, confirming the value of these targets that had been identified previously in Table 4. Table 6 summarizes the various targets identified through studies of both renal and cardiac grafts. FIG. 6 provides an expanded summary of that depicted in FIG. 5, reflecting results of the additional analysis.

TABLE 5
Targets identified and grouped through additional analysis

| Target | Group | Predictive | Sort Independently | Newly Identified |
|---|---|---|---|---|
| ENO1 | 1/2 | X | X | |
| AGRN | 1/2 | X | X | |
| EMCN | 1/2 | X | X | X |
| SSB | 2 | | X | X |
| Actin | 3 | | | |
| FLT3LG | 3 | | | |
| PRKCH | 3 | | | |
| IL-21 | 3 | | | |

Group 1 = Core + Predictive in the CART Analysis
Group 2 = Sort Independently + UCLA Newly Described
Group 3 = Cluster Together in the Correlation Matrix

TABLE 6
All targets identified

| Additional | | | |
|---|---|---|---|
| Analysis | Independent Analysis | | |
| Cardiac, Adult | Cardiac, Adult | Renal, Pediatric | Renal, Adult |
| ENO1*^{,} ** | Tubulin | DEXI* | Tubulin |
| AGRN*^{,} ** | LPHN1* | LGALS3* | SHC3*** |
| EMCN*^{,} ** | TG*** | SNPRN* | CXCL11*** |
| SSB** | GAPDH*** | CSF2** | |
| Actin*** | FN1*** | IL-8*** | |
| FLT3LG*** | NPHS1*** | STAT6** | |
| PRKCH*** | VIM*** | LGALS8** | |
| IL-21*** | Myosin*** | KRT18* | |
| | VCL** | KRT8* | |
| | PECR*** | GSTT1*^{,} ** | |
| | | LMNA*** | |
| | | Collagen II* | |
| | | ATP5B*** | |
| | | SNRPB2* | |
| | | PLA2R1*^{,} ** | |

Italics indicates newly identified.
Bold face indicates core (across organs).
*Indicates predictive in CART analysis.
**Indicates sorts independently.
***Indicates cluster together in correlation matrix.

### REFERENCES

Fan, L. et al. Neutralizing IL-17 prevents obliterative bronchiolitis in murine orthotopic lung transplantation. Am J Transplant 11, 911-22 (2011).

Haas, M., et al, Banff meeting report writing (2014). “Banff 2013 meeting report: inclusion of c4d-negative antibody-mediated rejection and antibody-associated arterial lesions.” Am J Transplant 14(2): 272-283.

Huang da, W., B. T. Sherman and R. A. Lempicki (2009). “Bioinformatics enrichment tools: paths toward the comprehensive functional analysis of large gene lists.” Nucleic Acids Res 37(1): 1-13.

Michaels, P. J., et al. (2003). “Humoral rejection in cardiac transplantation: risk factors, hemodynamic consequences and relationship to transplant coronary artery disease.” J Heart Lung Transplant 22(1): 58-69.

Pearl, M. H., et al. (2016). “Bortezomib may stabilize pediatric renal transplant recipients with antibody-mediated rejection,” Pediatr Nephrol 31(8): 1341-1348.

Simko, T. w. a. V. (2017). “R package “corrplot”: Visualization of a Correlation Matrix (Version 0.84).”

Smith, N. R. D. a. H. (1998). “Applied Regression Analysis.”

Stewart, S., et al. (2005). “Revision of the 1990 working formulation for the standardization of nomenclature in the diagnosis of heart rejection.” J Heart Lung Transplant 24(11): 1710-1720.

Zhang, Q., et al, (2005). “Development of posttransplant antidonor HLA antibodies is associated with acute humoral rejection and early graft dysfunction.” Transplantation 79(5): 591-598.

Zhang, Q. and E. F. Reed (2016). “The importance of non-HLA antibodies in transplantation.” Nat Rev Nephrol 12(8): 484-495.

What is claimed is:

1. A composition comprising a collection of solid-phase substrates coated with one or more homogenous populations of binding agents, wherein each homogenous population of binding agents specifically binds to an antibody that is directed against a single antigen selected from the group consisting of: dexamethasone-induced transcript (DEXI), C—X—C motif chemokine ligand 11 (CXCL11), cytokeratin 18 (KRT18), cytokeratin 8 (KRT8), Tubulin, including tubulin alpha 1 b (also referred to as TUBα1b or TUBA1B), latrophilin 1 (LPHN1), Colony stimulating factor 2 (CSF2), Signal Transducer And Activator Of Transcription 6 (STATE), lectin galactoside-binding soluble 3 (LGALS3), SHC Adaptor Protein 3 (SHC3), Glyceraldehyde-3-phosphate dehydrogenase (GAPDH), Glutathione 5-Transferase theta-1 (GSTT1), phospholipase A2 receptor 1 (PLA2R1), Interleukin 8 (IL-8), lectin galactoside-binding soluble 8 (LGALS8), Small Nuclear Ribonucleoprotein Polypeptide N (SNPRN), Myosin, Peroxisomal trans-2-enoyl-CoA Reductase (PECR), vimentin (VIM), ATP synthase H+transporting mitochondrial F1 complex beta polypeptide (ATP5B), Collagen II, Prelamin-A/C (LMNA), small nuclear ribonucleoprotein polypeptide B (SNRPB2), fibronectin 1 (FN1), nephrosis 1, congenital, Finnish type (NPHS1), Thyroglobulin (TG), and Vinculin (VCL).

2. The composition of claim 1, wherein the collection of solid-phase substrates further comprises one or more additional homogenous populations of binding agents, wherein each additional homogenous population of binding agents specifically binds to an antibody that is directed against a single additional antigen selected from the group consisting of:
Alpha-enolase (ENO1), Agrin (AGRN), Endomucin (EMCN), Sjogren syndrome antigen B (SSB), Actin, fms-related tyrosine kinase 3 ligand (FLT3LG), Protein kinase C eta (PRKCH), and Interleukin 21 (IL-21).

3. The composition of claim 1 or 2, wherein the solid-phase substrates are porous or non-porous.

4. The composition of claim 2 or 3, wherein the solid-phase substrates comprise particles, nanoparticles, beads, nanobeads or microspheres.

5. The composition of 4, wherein the beads are polystyrene beads.

6. The composition of any one of the preceding claims, wherein the collection of solid-phase substrates comprises a microarray.

7. The composition of any one of the preceding claims, wherein the solid-phase substrates are fluorescently labeled, magnetically labeled, or radio labeled.

8. The composition of any one of the preceding claims, wherein the solid-phase substrates are labeled with a small molecule.

9. The composition of any one of the preceding claims, wherein the one or more homogenous populations of binding agents are conjugated to the surface of the solid-phase substrates.

10. The composition of claim 9, wherein the conjugation is covalent.

11. The composition of any one of the preceding claims, wherein the one or more homogenous populations of binding agents are attached to the surface of the solid-phase substrates by affinity.

12. The composition of any one of the preceding claims, wherein the binding agent is a polypeptide.

13. The composition of any one of the preceding claims, wherein the solid-phase substrates are coated with at least three different homogenous populations of binding agents that bind to at least three different antigens.

14. The composition of claim 13, wherein the substrates are coated with multiple different homogenous populations of binding agents that bind to the antibodies consisting of antibodies to Tubulin, LPHN1, SNRPN, KRT18, KRT8, LGALS3, SNRPB2, DEXI, Collagen II, PLA2R1, GSTT1, VCL, CSF2, LGALS8, and STAT6.

15. The composition of claim 13, wherein the substrates are coated with multiple different homogenous populations of binding agents that bind to the antibodies consisting of antibodies to Tubulin, LPHN1, TG, GAPDH, FN1, NPHS1, VIM, Myosin, VCL, and PECR.

16. The composition of claim 15, wherein the substrates are further coated with multiple different homogenous populations of binding agents that bind to the antibodies consisting of antibodies to ENO1, AGRN, EMCN, SSB, Actin, FLT3LG, PRKCH, and IL-21.

17. The composition of claim 13, wherein the substrates are coated with multiple different homogenous populations of binding agents that bind to the antibodies consisting of antibodies to DEXI, LGALS3, SNPRN, CSF2, IL-8, STAT6, LGALS8, KRT18, KRT8, GSTT1, LMNA, Collagen II, ATP5B, SNRPB2 and PLA2R1.

18. The composition of claim 13, wherein the substrates are coated with multiple different homogenous populations of binding agents that bind to the antibodies consisting of antibodies to Tubulin, SHC3 and CXCL11.

19. The composition of claim 13, wherein the substrates are coated with multiple different homogenous populations of binding agents that bind to the antibodies consisting of antibodies to DEXI, EMCN, SNRPN, LPHN1 and SSB.

20. The composition of claim 13, wherein the substrates are coated with multiple different homogenous populations of binding agents that bind to the antibodies consisting of antibodies to KRT18, GAPDH, AGRN, ENO1 and EMCN.

21. The composition of claim 13, wherein the substrates are coated with multiple different homogenous populations of binding agents that bind to the antibodies consisting of antibodies to Tubulin, LPHN1, SNRPN, KRT18, KRT8, LGALS3, SNRPB2, DEXI, Collagen II, GAPDH, ENO1, AGRN, EMCN, SSB, PLA2R1, GSTT1, VCL, CSF2, LGALS8, STAT6, IL-8, and SHC3.

22. The composition of claim 1, wherein the single antigen is selected from the group consisting of: LPHN1, TG, DEXI, CSF2, IL-8, LGALS3, SNRPN, STAT6, SHC3, and LGALS8.

23. The composition of claim 2, wherein the single additional antigen is selected from the group consisting of: EMCN and SSB.

24. The composition of any of the preceding claims, wherein at least one solid-phase substrate is detectably distinguishable from at least one other solid-phase substrate.

25. A method for determining the presence of one or more antibodies in a biological sample obtained from a subject, the method comprising:
a) contacting the biological sample with the composition of any of claims 1-24, and
b) detecting the binding of the one or more homogenous populations of binding agents to the one or more antibodies.

26. The method of claim 25, wherein the subject is a mammal.

27. The method of claim 26, wherein the subject is a human.

28. The method of any of claims 25-27, wherein the subject has received or will receive a heart or kidney transplant.

29. The method of claim 28, wherein the heart transplant is an allograft heart or kidney transplant.

30. The method of any of claims 25-29, wherein the biological sample is blood, plasma, serum, urine, spinal fluid, lymph fluid, synovial fluid, cerebrospinal fluid, tears, saliva, milk, mucosal secretion, effusion, sweat, biopsy aspirates, ascites or fluidic extracts.

31. The method of any of claims 25-30, wherein the detecting is by measuring a fluorescence intensity or by an immunological analysis.

32. A method for diagnosing a transplant rejection response in a subject that has undergone a heart or kidney transplant, the method comprising:
a) contacting a biological sample obtained from the subject with the composition of any of claims 1-24, and
b) measuring the levels of the one of more antibodies in the sample;
wherein increased levels of the one or more antibodies, compared to reference levels, indicates that the subject has developed a transplant rejection response in response to the heart or kidney transplant.

33. A method for predicting the likelihood of a transplant rejection response in a subject in need of a heart or kidney transplant, the method comprising:
a) contacting a biological sample from the subject with the composition of any of claims 1-24, and
b) measuring the levels of the one or more antibodies in the sample;
wherein increased levels of the one or more antibodies, compared to reference levels, indicates that the subject has an increased likelihood of developing a transplant rejection response after a heart or kidney transplant.

34. A method of treating a subject in need of treatment for a transplant rejection response after receiving a heart or kidney transplant, the method comprising:
a) contacting a biological sample obtained from the subject with the composition of any of claims 1-24,
b) measuring levels of the one or more antibodies in the sample, and
c) administering a treatment for transplant rejection to the subject when there are increased levels of the one or more antibodies, compared to reference levels of the one or more antibodies.

35. A kit comprising:
a) the composition of any of claims 1-24, and
b) reagents for detecting the binding of the one or more homogenous populations of binding agents to the antibodies.

36. The kit of claim 35, further comprising one or more reference samples.
